(19) United States
(12) Patent Application Publication
(10) Pub. No.: US 2014/0200489 A1
(43) Pub. Date: Jul. 17, 2014

(54) METHOD AND SYSTEM FOR TISSUE MODULATION

(71) Applicants: Boaz Behar, Ganei-Tikva (IL); Yoni Hertzberg, Moshav Ben-Shemen (IL); Avishai Henley, Hod-HaSharon (IL); Safrir Patt, Ramat-HaSharon (IL)

(72) Inventors: Boaz Behar, Ganei-Tikva (IL); Yoni Hertzberg, Moshav Ben-Shemen (IL); Avishai Henley, Hod-HaSharon (IL); Safrir Patt, Ramat-HaSharon (IL)

(73) Assignee: Perseus-Biomed Inc, Orangeburg, NY (US)

(21) Appl. No.: 14/342,395

(22) PCT Filed: Sep. 2, 2012
(86) PCT No.: PCT/IB2012/054525

§ 371 Date: Mar. 3, 2014

Related U.S. Application Data
(60) Provisional application No. 61/605,237, filed on Mar. 1, 2012.
(60) Provisional application No. 61/529,936, filed on Sep. 1, 2011.

(51) Int. Cl. A61N 7/00

(52) U.S. Cl. CPC A61N 7/00
USPC 601/3

(57) ABSTRACT

A method of modulating tissue of an internal organ in vivo is disclosed. The method comprises: fixating the tissue on a shaped device so as to shape the tissue generally according to a shape of the device; and focusing radiation on the fixated tissue using a radiation-emitting system so as to modulate the tissue, wherein the radiation-emitting system is non-local with respect to the shaped device.

### RELATED APPLICATIONS

This application claims priority from U.S. Provisional Patent Application No. 61/529,936 filed Sep. 1, 2011, and U.S. Provisional Patent Application No. 61/605,237 filed Mar. 1, 2012. Both documents are incorporated herein by reference in their entirety.

### FIELD AND BACKGROUND OF THE INVENTION

The present invention, in some embodiments thereof, relates to medical devices and techniques and, more particularly, but not exclusively, to a method and system useful for tissue modulation by delivering energy to the tissue or removing energy from the tissue.

There are many medical situations where tissue modulation or damage has been demonstrated to be clinically beneficial. Device-based approaches for tissue modulation include treatment of tissue by energy treatments, such as high intensity focused ultrasound (HIFU), cryotherapy, and treatment of tissue by electromagnetic radiation of various spectra, including X-Ray, microwave, radiofrequency (RF) and the like.

Tissue modulation treatments have heretofore been employed for many types of conditions and pathologies. Known in the art are thermal treatments of the prostate, and reduction of the nerve activity (also known as denervation) in cases of hyperactivity of the sympathetic nervous system.

For example, it has been demonstrated (Renal Sympathetic-Nerve Ablation for Uncontrolled Hypertension, New England Journal of Medicine, 2009; 361:932-934, Aug. 27, 2009) that denervation by energy treatment of renal artery nerves, can reduce blood pressure in uncontrolled hypertension patients. It is believed that such a treatment has additional clinical benefits, such as increasing insulin uptake.

U.S. Published Application No. 20070129760 the contents of which are hereby incorporated by reference, describes a technique in which a neural denervation element is positioned within a blood vessel of a patient, and activated to denervate the tissue that is innervated by neural matter located within or in proximity to the blood vessel. The neural denervation element is configured to deliver thermal energy, high intensity focused ultrasound (HIFU) or neuromodulatory agent to the neural tissue.

U.S. Pat. No. 5,492,126 the contents of which are hereby incorporated by reference, describes combining ultrasound image guidance with HIFU treatment for achieving improved accuracy in the treatment of tissue.

U.S. Pat. No. 6,576,875 the contents of which are hereby incorporated by reference, describes a method of ultrasound spectral analysis imaging of the treatment beam correlated with the tissue imaging to allow imaging of both tissues and beam.

U.S. Pat. No. 6,128,522 the contents of which are hereby incorporated by reference, describes an MRI guidance method for heat treatment methods such as HIFU.

Additional background art includes U.S. Pat. No. 7,630,750.

### SUMMARY OF THE INVENTION

According to an aspect of some embodiments of the present invention there is provided a method of modulating tissue of an internal organ in vivo. The method comprises: fixating the tissue on a shaped device so as to shape the tissue generally according to a shape of the device; and focusing radiation on the fixated tissue using a radiation-emitting system so as to modulate the tissue, wherein the radiation-emitting system is non-local with respect to the shaped device.

According to some embodiments of the invention the method wherein the radiation-emitting system is a non-invasive radiation-emitting system.

According to some embodiments of the invention the method wherein the radiation-emitting system is a minimally-invasive radiation-emitting system introduced into an organ other than the organ hosting the shaped device.

According to some embodiments of the invention the radiation comprises high intensity focused ultrasound (HIFU).

According to some embodiments of the invention the radiation selected from the group consisting of X-ray and microwave.

According to some embodiments of the invention the method further comprises scanning the focused radiation along a predetermined path corresponding to the shape of the device so as to form a modulation pattern on the tissue.

According to some embodiments of the invention the scanning comprises moving the radiation-emitting system.

According to some embodiments of the invention the scanning is effected by a phased array radiation-emitting system.

According to some embodiments of the invention the method further comprises receiving signals indicative of a relative position of the radiation-emitting system with respect to the shaped device, wherein the scanning is responsively to the relative position.

According to some embodiments of the invention the method further comprises sensing the radiation at or in proximity to the shaped device, and correcting the path responsively to the sensing.

According to some embodiments of the invention the sensing is performed selectively at a plurality of discrete locations.

According to some embodiments of the invention the sensing comprises reflecting the radiation outwardly and collecting the reflected radiation outside the body.

According to some embodiments of the invention the method comprises modulating the reflected radiation so as to encode spatial information therein.

According to some embodiments of the invention the method further comprises operating a data processor to execute an image analysis procedure so as to identify focal regions corresponding to the focused radiation.

According to some embodiments of the invention the imaging is performed intracorporeally.

According to some embodiments of the invention the method further comprises calibrating the radiation responsively to the sensing.

According to some embodiments of the invention the method comprises receiving prerecorded calibration data having a plurality of entries, each entry comprises a set of radiation parameters associated with a three-dimensional coordinate, and searching the data for three-dimensional coordinate corresponding to a sensing location to extract a respective set of radiation parameters, wherein the calibrating is also based on the respective set of radiation parameters.

According to some embodiment of the invention the method comprises receiving prerecorded calibration data having a plurality of entries, each entry comprises a set of radiation parameters associated with a three-dimensional coordinate, and searching the data for radiation parameters received by sensor, for its corresponding three-dimensional coordinate.

According to some embodiments of the invention the method further comprises, prior to the modulation of the tissue, operating the radiation-emitting system to emit non-damaging radiation, wherein the correction of the path is performed during the emission of the non-damaging radiation.

According to some embodiments of the invention the method comprises, prior to the modulation of the tissue, operating the radiation-emitting system to emit non-damaging radiation, wherein the calibration is performed during the emission of the non-damaging radiation.

According to some embodiments of the invention the method comprises repeating the emission of the non-damaging radiation and the modulation intermittently.

According to some embodiments of the invention at least one of: a rate and a duty cycle of the intermittent repetition is selected to match one of a characteristic breathing cycle of a subject having the organ, a heartbeat, movement of a digestive organ, a patient movement, or a combination of any these.

According to some embodiments of the invention the predetermined path forms a non-closed loop spanning, optionally in a helical pattern, about a longitudinal axis, and optionally spanning between 90° and 540°.

According to some embodiments of the invention the predetermined path generally forms a helix.

According to some embodiments of the invention the tissue is a nerve and the modulation comprises denervation. According to some embodiments of the invention the nerve is a part of an autonomic nervous system. According to some embodiments of the invention the nerve is selected from the group consisting of a nerve leading to a kidney, a sympathetic nerve connected to a kidney, an afferent nerve connected to a kidney, an efferent nerve connected to a kidney, a renal nerve, a renal sympathetic nerve at a renal pedicle, a nerve trunk adjacent to a vertebra, a ganglion adjacent to a vertebra, a dorsal root nerve, an adrenal gland, a motor nerve, a nerve next to a kidney, a nerve behind an eye, a celiac plexus, a nerve within a vertebral column, a nerve around a vertebral column, nerve extending to a facet joint and a celiac ganglion. According to some embodiments of the invention the nerve a renal artery nerve.

According to some embodiments of the invention the tissue is a prostatic tissue in the prostate.

According to some embodiments of the invention the organ is selected from the group consisting of prostate, liver, kidney, pancreas and heart.

According to an aspect of some embodiments of the present invention there is provided a catheter system. The system comprises: a shaped device adapted for being introduced into a living body and being configured for fixating a tissue thereon so as to shape the tissue generally according to a shape of the device; and at least one passive ultrasound sensor mounted on the device and configured for sensing at least one of: a position of the shaped device within a living body, and radiation emitted by an ultrasound radiation-emitting system which is non-local with respect to the device and which is optionally external to the body. Additionally or alternatively, the system comprises a device suitable for being positioned in tissue and expandable to a generally known shape of the tissue.

According to some embodiments of the invention there is provided a shaped device adapted for being introduced into a living body and being configured for fixating a tissue thereon so as to shape the tissue generally according to a shape of the device; and at least one passive ultrasound sensor mounted on the device and configured for sensing at least one of: a position of the shaped device within a living body, and radiation emitted by an ultrasound radiation-emitting system non-local with respect to the device and optionally external to the body.

According to some embodiments of the invention the system wherein the radiation comprises high intensity focused ultrasound (HIFU).

According to an aspect of some embodiments of the present invention there is provided a system for modulating tissue of an internal organ in vivo. The system comprises: a shaped device adapted for being introduced into a living body and being configured for fixating a tissue thereon so as to shape the tissue generally according to a shape of the device; a radiation-emitting system configured for emitting radiation from a location external to the body and focusing the radiation on the fixated tissue; a scanning system operative to scan the radiation over the fixated tissue; and a controller, configured for controlling the radiation-emitting system and the scanning system such that the scan is along a predetermined path corresponding to the shape of the device so as to from a modulation pattern on the tissue.

According to some embodiments of the invention the system comprises at least one sensor mounted on the shaped device and configured for sensing at least one of: a position of the shaped device within a living body, and radiation emitted by the radiation-emitting system, wherein the controller is configured for receiving signals from the at least one sensor and for controlling the radiation-emitting system and the scanning system, responsively to the signals.

According to some embodiments of the invention the at least one sensor comprises a plurality of sensors arranged at a plurality of discrete locations over the shaped device.

According to some embodiments of the invention the system comprises: at least one reflector mounted on the shaped device and configured for reflecting radiation emitted by the radiation-emitting system; and at least one radiation sensor configured for sensing the reflected radiation at one or more sensing locations external to the shaped device; wherein the controller is configured for receiving signals from the at least one radiation sensor and for controlling the radiation-emitting system and the scanning system, responsively to the signals.

According to some embodiments of the invention the at least one radiation sensor comprises a sensors adapted to be located outside the body.

According to some embodiments of the invention the at least one radiation sensor comprises a sensors adapted to be located inside the body but external to the device.

According to some embodiments of the invention the reflectors are configured for modulating the reflected radiation to encode spatial information therein, wherein the controller is configured for extracting the spatial information from the signals, and for controlling the radiation-emitting system and the scanning system responsively to the extracted spatial information.

According to some embodiments of the invention the controller is configured for calibrating the radiation responsively to the sensing.

According to some embodiments of the invention the controller is configured to access prerecorded calibration data having a plurality of entries, each entry comprises a set of radiation parameters associated with a three-dimensional coordinate, and to search the data for three-dimensional coordinate corresponding to a sensing location so as to extract a respective set of radiation parameters, wherein the calibrating is also based on the respective set of radiation parameters.

According to some embodiments of the invention the controller is configured to access prerecorded calibration data having a plurality of entries, each entry comprises a set of radiation parameters associated with a three-dimensional coordinate, and to search the data for radiation sensing parameters to determine its corresponding three-dimensional coordinates.

According to some embodiments of the invention the system comprises: an intracorporeal imaging system configured for imaging the fixated tissue and regions in proximity thereto, wherein the controller is configured for analyzing imagery data received from the intracorporeal imaging system, and identifying focal regions corresponding to the focused radiation.

According to an aspect of some embodiments of the present invention there is provided a system for modulating tissue of an internal organ in vivo. The system comprises: a shaped device adapted for being introduced into a living body; a radiation-emitting system configured for emitting radiation from a location external to the body and focusing the radiation on the shaped device; at least one sensor mounted on the device, and being configured for sensing the radiation; and a data processor, configured for analyzing signals received from the at least one sensor and calculate at least one of: a relative location and a distance of a focal region of the radiation.

According to some embodiments of the invention the system wherein the radiation-emitting system configured for scanning the tissue and wherein the data processor is also configured for calculating a scanning path of the focal region.

According to some embodiments of the invention the data processor is configured for receiving a geometric relationship between the at least one sensor and a shape of the tissue, and to calculate the relative location and/or the distance based, at least in part, on the geometric relationship.

According to some embodiments of the invention the data processor is configured for calculating geometric relationship between the at least one sensor and a shape of the tissue, and to calculate the relative location and/or the distance based, at least in part, on the geometric relationship.

According to some embodiments of the invention a system includes multiple sensors mounted on multiple devices positioned in vivo, and a processor configured for calculating a treatment path of a beam to fit a geometry according to which the beam will not harm tissue located at position known relative to these multiple sensors.

According to an aspect of some embodiments of the present invention there is provided a method of directing a tissue-modulating energy beam from a source distant from a target tissue to treat the target tissue in a body of a patient, comprising:
a) positioning within a body and near a target tissue at least one sensor operable to detect energy from the energy source;
b) aiming towards a plurality of positions within an intrabody volume containing the at least one sensor a plurality of experimental energy beams of non-damaging intensity, each beam being aimed according to at least one experimental beam-aiming parameter used to aim the each beam;
c) selecting, from among the plurality of experimental beam-aiming parameters, a parameter shown by detection of beam energy at the sensor to have aimed its associated experimental energy beam towards the sensor; and
d) aiming a tissue-modulating treatment energy beam from the source toward the target tissue according to a treatment-beam-aiming parameter calculated as a function of the selected experimental beam-aiming parameter.

According to some embodiments of the invention the source distant from the target tissue is positioned outside a body.

According to some embodiments of the invention the aiming of the plurality of experimental energy beams comprises scanning the non-damaging energy beam over the intrabody volume by successively modifying at least one beam-aiming parameter to affect aiming of the experimental beams, while monitoring beam energies received at the at least one sensor.

According to some embodiments of the invention selecting the selected parameter comprises selecting from among the plurality of experimental beam-aiming parameters a parameter whose aimed beam produced within 5% of the maximum energy detected at the sensor for the group of experimental beams.

According to some embodiments of the invention the tissue-modulating beam is aimed towards the sensor.

According to some embodiments of the invention the tissue-modulating beam is aimed towards a position at a calculated displacement from a position of the sensor.

According to some embodiments of the invention further comprises positioning near the target tissue a plurality of sensors, and identifying beam-aiming parameters which aim beams at each of the plurality of sensors.

According to some embodiments of the invention at least one of the sensors is a pressure sensor and the energy source is a high intensity focused ultrasound (HIFU) projector.

According to some embodiments of the invention the sensor is an x-ray sensor and the energy source is a source of x-rays.

According to some embodiments of the invention the method comprising identifying the beam-aiming parameter by successively modifying beam aiming along a first and then along a second Cartesian coordinate, and detecting separately for each Cartesian coordinate which beam-aiming parameters maximize energy received at the sensor.

According to some embodiments of the invention further comprising modifying beam aiming along a third Cartesian coordinate, and detecting which beam-aiming parameters maximize energy received at the sensor.

According to some embodiments of the invention further comprising identifying the beam-aiming parameter by successively modifying beam aiming according to first and then second coordinates in a spherical coordinate system, detecting separately for each coordinate which beam-aiming parameters maximize energy received at the sensor.

According to some embodiments of the invention further comprising detecting a delay between emission of the beam at the energy source and detection of the beam at the sensor.

According to some embodiments of the invention further comprising cyclically repeating the measurement phase of activity followed by the treatment phase of activity in a repeating cycle.

According to some embodiments of the invention the measurement phase of activity occupies between 0.1% and 30% of each of the cycles.

According to some embodiments the invention further comprises utilizing differences between identified parameters of two different sensors at a known distance from each other to calculate a second set of bean-aiming parameters which, when used to project a beam, will displace the beam from the sensors by a pre-calculated amount.

According to some embodiments of the invention the sensors are mounted on a catheter in a blood vessel, and the calculated beam-aiming parameters are calculated to direct a beam to a position near the blood vessel and distanced from the sensors.

According to some embodiments of the invention the method further comprises repeating the measurement phase of activity alternating with the treatment phase of activity to direct the tissue-modulating beam at a moving treatment target.

According to some embodiments of the invention the sensor is mounted on a shaped device operable to fixate position of the target tissues.

According to some embodiments of the invention the sensor is mounted on a shaped device expandable to at least approximately match size and shape of at least a part of the target tissue.

According to some embodiments of the invention further comprising calculating a series of beam-aiming parameters which displaces the tissue-modulating beam in a pre-planned pattern over an extended surface of the tissue.

According to some embodiments of the invention further comprising beaming energy from a plurality of energy sources.

According to some embodiments of the invention the plurality of energy sources comprises a phased array.

According to some embodiments of the invention the phased array is a high intensity focused ultrasound (HIFU) projection array.

According to some embodiments of the invention further comprising separately measuring a relationship between beam generation parameters and energy detected by at least one sensor, for each of a plurality of transmitting elements.

According to some embodiments of the invention further comprising distinguishing between energies originating simultaneously at a plurality of sources during the measurement cycle by modulating energies transmitted from at least some of the sources, and detecting the modulation in responses of the sensor, identifying a transmission source from which a modulated energy originated according to the detected modulation.

According to some embodiments of the invention the modulation is a frequency modulation.

According to some embodiments of the invention a plurality of transmitting elements are fired at different times during a same measurement phase of activity.

According to some embodiments of the invention further comprising firing a plurality of transmitting elements in a known order during a same measurement phase of activity, and relating energy signals received at the sensor to particular originating transmission elements according to an order in which the energy signals are detected.

According to some embodiments of the invention further comprising measuring a delay between transmission of an energy beam and detection of the beam at the sensor, for a plurality of energy sources.

According to some embodiments of the invention further comprising firing, during the treatment phase of activity, a plurality of elements of a phased array, the firing occurring in a timed sequence whose timing is calculated based on delays detected during the measurement phase of activity.

According to some embodiments of the invention further comprising using energy beams at the non-damaging intensity from each of the plurality of energy sources to identify those sources from which projected energy is detected by a sensor near the target tissue, and aiming the tissue-modulating energy beam towards the target tissue only from those energy sources from which energy of the non-damaging intensity was successfully detected by the sensor.

According to some embodiments of the invention further comprising cyclically alternating between sequentially identifying beam-aiming parameters for a plurality of energy sources and delivering tissue-modulating energy to a target tissue from a plurality of sources.

According to some embodiments of the invention the sequentially identifying of the beam-aiming parameters comprises a sequence of simultaneous firings each of a plurality of adjacent energy sources.

According to some embodiments of the invention further comprising using a plurality of sensors at least some of which are located within different body organs.

According to some embodiments of the invention one of the different body organs is an esophagus and another of the different body organs is a heart.

According to some embodiments of the invention one of the different body organs is a renal artery and another of the different body organs is a renal vein.

According to some embodiments of the invention at least one of the sensors is in an esophagus and mounted on an inflatable balloon.

According to an aspect of some embodiments of the present invention there is provided a method for protecting an esophagus from damage during energy treatment of a nearby organ, comprising introducing into the esophagus an expandable device which comprises a magnetic element, expanding the inflatable device to fix the device within the esophagus, and utilizing an extracorporeal magnet to move the magnetic element, thereby moving the device, thereby moving the esophagus to a position where it is at least partially protected from an energy treatment applied to a nearby organ.

According to some embodiments of the invention further comprising introducing into the esophagus a sensor able to detect energy from the energy treatment, and doing one of warning a physician and modifying the energy treatment when energy detected by the sensor exceeds a predetermined amount.

According to an aspect of some embodiments of the present invention there is provided a system for directing towards a target tissue in a body of a patient a tissue-modulating energy beam from an energy source distant from the target tissue, comprising:
a) a sensor operable to detect energy from the energy source and positionable within a body in a vicinity of the target tissue;
b) an energy projector distant from the target tissue;
c) a projection controller programmed modify energy beam aiming parameters used by the energy projector as a function of energy detection reports issued by the sensor.

According to some embodiments of the invention the energy projector is outside the body of a patient.

According to some embodiments of the invention the controller is programmed to command projecting of non-destructive energies in a variety of directions while collecting information from the sensor during a measurement phase of activity, and to subsequently command projection of tissue-modulating energies during a treatment phase of activity, using tissue-modulating energy beam aiming parameters calculated as a function of the collected information.

According to some embodiments of the invention the tissue-modulating energy beam aiming parameters are calculated so as to aim the tissue-modulating energy beam towards the sensors during the treatment phase of activity.

According to some embodiments of the invention the tissue-modulating energy beam aiming parameters are calculated so as to aim the tissue-modulating energy beam towards a position at a known distance and direction away from the sensor during the treatment phase of activity.

According to some embodiments of the invention the aiming parameters are systematically modified during one or more of the treatment phases to aim energy in a pre-planned pattern having a known spatial relationship to the sensor.

According to some embodiments of the invention the pre-planned pattern is designed to avoid directing the tissue modulating energy towards a specific body organ.

According to some embodiments of the invention the sensor is mounted on a shaped device operable to fixate target tissues in a fixed position relative to the shaped device.

According to some embodiments of the invention the sensor is mounted on a shaped device operable to take the shape of at least a portion of a target tissue.

According to some embodiments of the invention the sensor is mounted on a shaped device operable to take the shape of a tissue structure which has a known geometry relative to target tissue.

According to some embodiments of the invention the system comprises a mechanism for displacing the shaped device and a tissue fixated thereon, within a body.

According to some embodiments of the invention the mechanism comprises a magnet.

According to some embodiments of the invention the sensor is mounted on a shaped device operable to fixate target tissues in a fixed position relative to the shaped device.

According to some embodiments of the invention the system further comprising a mechanism for moving an organ distinct from the target tissue away from the target tissue.

According to some embodiments of the invention the mechanism comprises a magnet.

According to some embodiments of the invention a portion of the mechanism is sized and shaped for insertion into an esophagus.

According to some embodiments of the invention a portion of the mechanism is sized and shaped for insertion into a body through a nasal canal.

According to some embodiments of the invention the mechanism comprises an expandable device which comprises a magnetic element.

According to some embodiments of the invention the mechanism comprises a sensor operable to detect heat.

According to some embodiments of the invention the mechanism comprises a sensor operable to detect beamed energy.

According to some embodiments of the invention, the system further comprising a sensor operable to detect at least one of
a) dangerous levels of heat in an organ; and
b) dangerous levels of beamed energy beamed to a position in or near the organ; and

further comprising a controller which receives signals from the sensor and is programmed to modify an energy treatment upon receipt of a signal from the sensor reporting a dangerous condition.

According to some embodiments of the invention, the system further comprising a shaped device operable to fixate a blood vessel and further comprising a mechanism for moving the blood vessel.

According to some embodiments of the invention the system comprises a plurality of sensors positioned in a vicinity of the target tissue.

According to some embodiments of the invention the sensor is a pressure sensor and the energy source is a high intensity focused ultrasound (HIFU) projector.

According to some embodiments of the invention the sensor is an x-ray sensor and the energy source is a source of x-rays.

According to some embodiments of the invention the controller is programmed to calculate a delay between projection of energy by the energy source and detection of the energy by the sensor.

According to some embodiments of the invention the energy source comprises a plurality of energy transmitters.

According to some embodiments of the invention the controller is programmed to calculate the delay for each of the plurality of energy transmitters.

According to some embodiments of the invention the controller is programmed to calculate the delay for each of a plurality of energy transmitters whose delays were not measured during a previous measurement cycle, by extrapolating data from energy transmitters whose delays were measured during the previous measurement cycle.

According to some embodiments of the invention the energy source comprises at least one phased array of transmitters.

According to some embodiments of the invention the controller is programmed to project tissue-modifying energies during a treatment phase of activity only from energy transmitters whose transmissions of lower levels of energy during a measurement phase of activity were detected by the sensor.

According to some embodiments of the invention the controller is programmed to cyclically alternate between a measurement phase of activity during which non-destructive energies are projected and a treatment phase of activity during which tissue-modulating energies are projected.

According to some embodiments of the invention the cycles of the cyclical activity are of between 10 milliseconds and 200 milliseconds duration.

According to some embodiments of the invention the measurement phase utilizes between 0.1% and 30% of the cycle duration.

Unless otherwise defined, all technical and/or scientific terms used herein have the same meaning as commonly understood by one of ordinary skill in the art to which the invention pertains. Although methods and materials similar or equivalent to those described herein can be used in the practice or testing of embodiments of the invention, exemplary methods and/or materials are described below. In case of conflict, the patent specification, including definitions, will control. In addition, the materials, methods, and examples are illustrative only and are not intended to be necessarily limiting.

Implementation of the method and/or system of embodiments of the invention can involve performing or completing selected tasks manually, automatically, or a combination thereof. Moreover, according to actual instrumentation and equipment of embodiments of the method and/or system of the invention, several selected tasks could be implemented by hardware, by software or by firmware or by a combination thereof using an operating system.

For example, hardware for performing selected tasks according to embodiments of the invention could be implemented as a chip or a circuit. As software, selected tasks according to embodiments of the invention could be implemented as a plurality of software instructions being executed by a computer using any suitable operating system. In an exemplary embodiment of the invention, one or more tasks according to exemplary embodiments of method and/or system as described herein are performed by a data processor, such as a computing platform for executing a plurality of instructions. Optionally, the data processor includes a volatile memory for storing instructions and/or data and/or a non-volatile storage, for example, a magnetic hard-disk and/or removable media, for storing instructions and/or data. Optionally, a network connection is provided as well. A display and/or a user input device such as a keyboard or mouse are optionally provided as well.

### BRIEF DESCRIPTION OF THE DRAWINGS

Some embodiments of the invention are herein described, by way of example only, with reference to the accompanying drawings. With specific reference now to the drawings in detail, it is stressed that the particulars shown are by way of example and for purposes of illustrative discussion of embodiments of the invention. In this regard, the description taken with the drawings makes apparent to those skilled in the art how embodiments of the invention may be practiced.

In the drawings:

FIG. 1 is a flowchart diagram of a method suitable for modulating tissue of an internal organ in vivo, according to some exemplary embodiments of the present invention;

FIG. 2 is a schematic illustration of renal nerves;

FIG. 3 is a flowchart diagram describing the method of the present embodiments in greater detail;

FIG. 4A is a schematic illustration of a catheter system, according to some embodiments of the present invention;

FIG. 4B is a schematic illustration of a shaped device according to some embodiments of the present invention;

FIG. 5 is a schematic illustration of a system for modulating tissue of an internal organ in vivo, according to some embodiments of the present invention;

FIG. 6A is a schematic illustration of a system for aiming an energy beam with reference to a sensor positioned within a body and near a target tissue, according to some embodiments of the present invention;

FIG. 6B is a flowchart providing additional details of a method of aiming energy towards a target tissue, according to some embodiments of the present invention;

FIG. 7A is a schematic illustration of a system for aiming an energy beam with reference to a plurality of sensors positioned within a body and near a target tissue, according to some embodiments of the present invention;

FIG. 7B is a flowchart providing details of a method of aiming energy from a phased array energy source towards a target tissue, according to some embodiments of the present invention, and

FIG. 8 is a simplified schematic of a system for protecting an organ during use of an energy beam to treat another organ.

### DESCRIPTION OF SPECIFIC EMBODIMENTS OF THE INVENTION

The present invention, in some embodiments thereof, relates to medical devices and techniques and, more particularly, but not exclusively, to a method and system useful for tissue modulation by delivering energy to the tissue or removing energy from the tissue.

Before explaining at least one embodiment of the invention in detail, it is to be understood that the invention is not necessarily limited in its application to the details of construction and the arrangement of the components and/or methods set forth in the following description and/or illustrated in the drawings and/or the Examples. The invention is capable of other embodiments or of being practiced or carried out in various ways.

It is recognized that the treatment of tissues by remote, focused, energy methods such as X-Ray radiation, microwave radiation, ultrasound radiation and alike, generally requires that the energy beam be directed and/or focused onto the tissue. It is desired that the direction of energy be inline with the position and shape of the organ or tissue to be treated, so as to effectively treat the tissue, preferably with minimal or no damage to neighboring tissues.

When using, for example, ultrasound such as, but not limited to, high intensity focused ultrasound (HIFU), the direction of the ultrasonic beam entering the body may not assure hitting the target tissue, since the ultrasonic energy passes through multiple tissue segments, experience multiple different ultrasonic speed, and therefore deflections in directions which are a priori unknown. Another cause for misalignment between the energy beam and the target tissue relates to tissue motion which results in continuous variation of the spatial relations between the tissue and the radiation system. Beam misalignment and unpredicted phase errors can cause focus dispersion and reduced treatment efficacy, because the more concentrated the energy is, the better the chances for successful denervation, but in dispersed beams, the available energy is spread over a larger area and weaken the therapeutic effect.

Heretofore, image guidance of HIFU has been utilized using MRI and ultrasound, typically tuning the direction and path of the energy beam at low, non harmful energy levels, and turning the power up for treatment once in position.

MRI can be used as a technique for measuring intrabody temperature noninvasively. When using MRI to guide HIFU for example, the HIFU beam can be tuned to a low, non harmful energy level, and the MRI image can image the tissue shape and position, as well as the beam focus via the temperature imaging capability of MRI.

It was found by the present inventors, that conventional MRI guided heat treatments have several drawbacks. For example, for use in real time, all of the equipment used within the procedure needs to be non ferromagnetic, substantially increasing the complexity and price of auxiliary equipment. Additionally, MRI has a relatively slow shutter time, causing blurriness over moving organs such as arteries and veins in the vicinity of the lungs.

Traditional diagnostic ultrasound is known to be suitable for imaging the target tissue location and shape. The present inventor recognized that this modality is not capable of imaging the HIFU beam because this beam does not create an echo which is different from the tissue it passes through. Known techniques for imaging the HIFU beam include: imaging of mechanical artifacts of the beam, analysis of speckles, and spectral analysis.

It was found by the present inventors, that conventional ultrasound guided heat treatments have several drawbacks. For example, when the guidance is based on mechanical artifacts, it is necessary to induce such artifacts, which are oftentimes not desirable, in particular when the beam interacts with tissues other that the target tissue. When the guidance is based on imaging of speckles or spectral analysis, the spatial resolution of the location of the beam is confined to 3-10 wavelengths. This results in a bad resolution, or requires very high frequencies which reduce the effective imaging range.

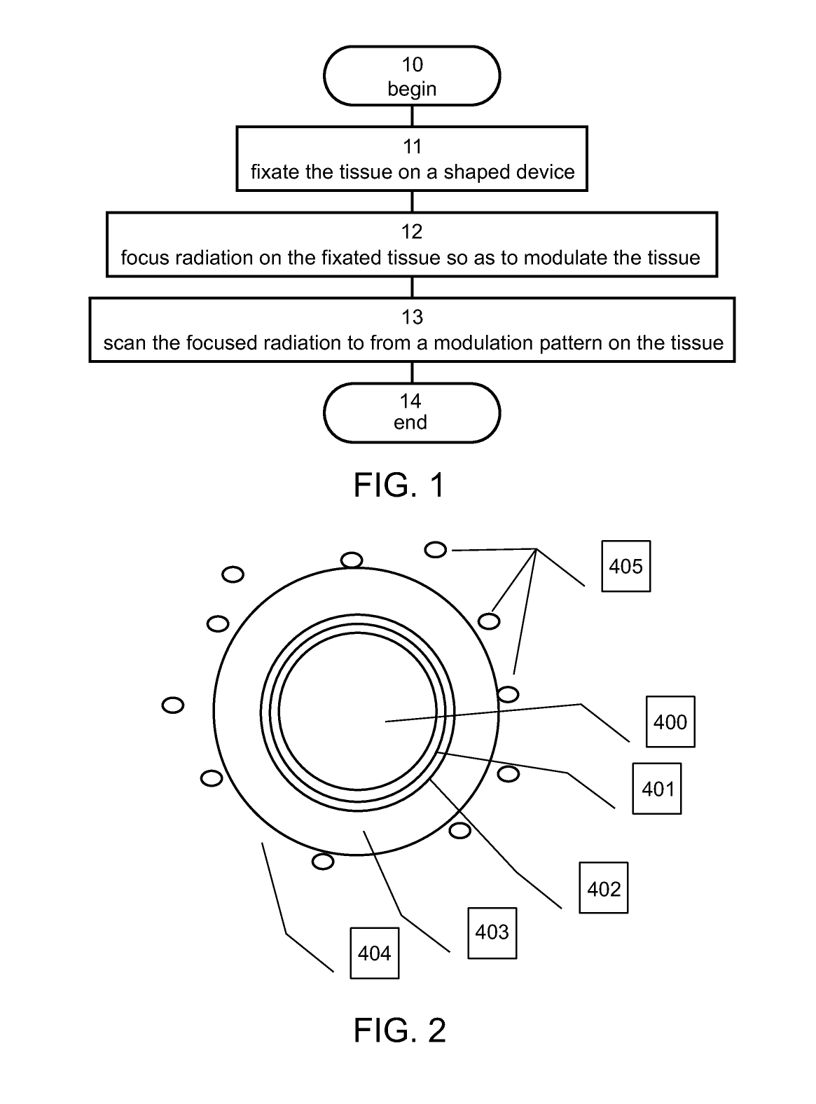

Referring now to the drawings, FIG. 1 is a flowchart diagram of a method suitable for modulating tissue of an internal organ in vivo, according to some embodiments of the present invention. It is to be understood that, unless otherwise defined, the operations described hereinbelow can be executed either contemporaneously or sequentially in many combinations or orders of execution. Specifically, the ordering of the flowchart diagrams is not to be considered as limiting. For example, two or more operations, appearing in the following description or in the flowchart diagrams in a particular order, can be executed in a different order (e.g., a reverse order) or substantially contemporaneously. Additionally, several operations described below are optional and may not be executed.

The method begins at 10 and continues to 11 at which a target tissue is fixated on a shaped device so as to shape the tissue generally according to a shape of the device.

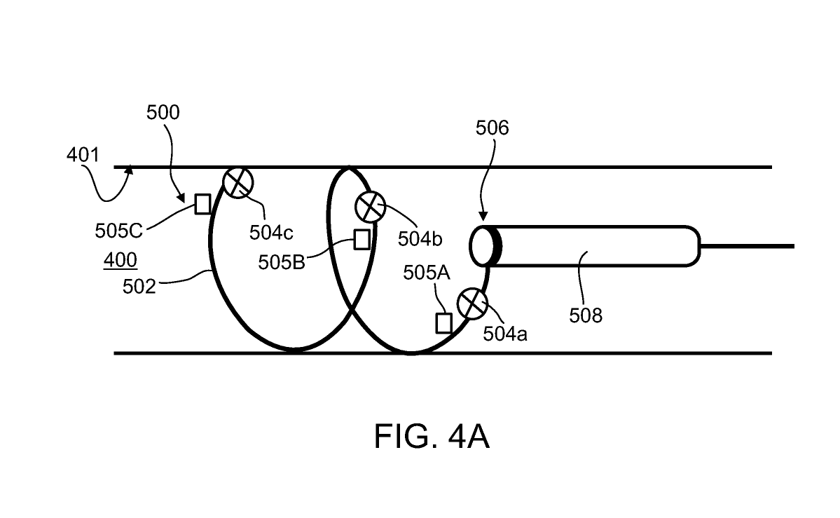

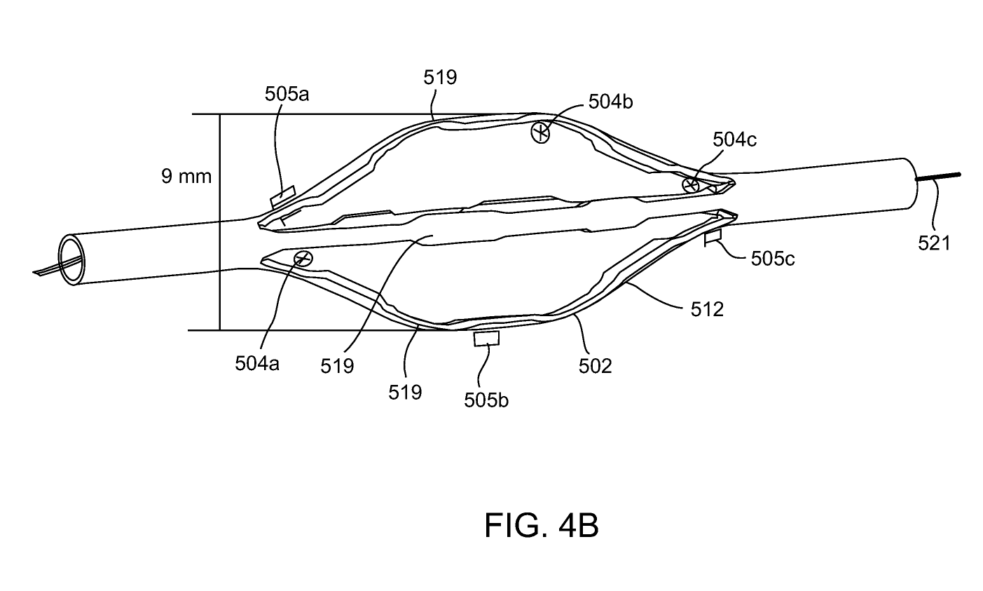

The term “fixated” is used herein to specify a condition in which a tissue is immobilized with respect to a shaped device. For example, a device such as that shown in FIG. 4B may fixate a tissue by expanding within that tissue (e.g. an artery) until that tissue is pressured or somewhat stretched by the device, and is thereby immobilized. By this process the tissue may also be constrained into a predetermined shape corresponding to the shape of the shaped device. Any other method of attaching a tissue to a device is also be considered as ‘fixating’ that tissue with respect to the device. However, it is noted here that altering the geometry of the tissue being ‘fixated’ is not a requirement of the invention; any arrangement which immobilizes one with respect to the other is to be understood as included in the meaning of “fixated” as that term is used in the this disclosure and in the accompanying claims. For example, a device which expands within a lumen so as to assume a known geometrical shape of that lumen would be considered “fixated”, as that term is used herein, if lumen and expandable device were thereby temporarily immobilized one with respect to the other, even if both are moving in absolute space, e.g. as a result of a heartbeat or respiration. In some embodiments, a shaped device is an expandable device operable to expand to at least approximately match a shape and size of at least a part of said tissue.

Note also that the terms the terms “energy projector” and “energy transmitter” and “energy emitter”, as used herein, are considered to have the same meaning and are used interchangeably.

The target tissue may be any type of internal tissue. Representative examples include, without limitation, a nerve tissue, particularly a nerve which is a part of an autonomic nervous system, a prostate tissue, a liver tissue, a kidney tissue, a pancreas tissue and a heart tissue.

Nerve tissues suitable for some embodiments of the present invention include, without limitation, a nerve leading to a kidney, an efferent nerve leading from the kidney, a renal nerve, a sympathetic nerve connected to a kidney, an afferent nerve connected to a kidney, a renal sympathetic nerve at a renal pedicle, a nerve trunk adjacent to a vertebra, a ganglion adjacent to a vertebra, a dorsal root nerve, an adrenal gland, a motor nerve, a nerve next to a kidney, a nerve behind an eye, a celiac plexus, a nerve within a vertebral column, a nerve around a vertebral column, a nerve extending to a facet joint, a celiac ganglion, a cardiac nerve, a portion of a brain. Some embodiments of the invention also treat cancerous tissue in or near a lumen, such as for example a blood vessel, in which a catheter may be placed.

In some embodiments of the present invention the tissue is a renal artery nerve, and in some embodiments of the present invention is the tissue is a renal vein nerve.

The shaped device is preferably adapted for being introduced into the body, for example, in an endoscopic, laparoscopic or intravascular manner. Typically, the shaped device is provided as, or being mounted on, a catheter system, which may be endoscopic, laparoscopic or intravascular. The shaped device may have any shape that the tissue can assume. For example, the device may have a generally cylindrical shape (e.g., a cylindroid, a circular cylinder etc.). Other shapes include, without limitation, a spiral, a helix, a disk, an oval, a cuboid, a prism, a sphere, a hemisphere, a portion of a sphere, a spheroid, a portion of a spheroid, a prolate spheroid, an oblate spheroid, an ellipsoid, a portion of ellipsoid, a hyperboloid, a portion of a hyperboloid, a paraboloid, a portion of a paraboloid, a cylindrical shell, a portion of a cylindrical shell, a polyhedron shell, a portion of a polyhedron shell, and any combination between two or more of these shapes.

The tissue may be fixated on the device by any technique known in the art. For example, the shaped device may be made expandable, wherein its expanded shape is preplanned. The device may assume its expanded shape in response to external activation (e.g., mechanical, thermal and/or electrical activation).

A representative example is an expandable mesh (e.g., a stent or the like) that upon expansion shapes the blood vessel co-axially to a catheter. (An example of such a structure is discussed hereinbelow with respect to FIG. 4B.) Also contemplated are embodiments in which the size and/or shape of the device is adapted to the target tissue of the specific subject. In these embodiments, the method first receives data pertaining to the size and/or shape of the target tissue in its relaxed state, and determines the shape of the device based on the received data. For example, when the target tissue is a blood vessel, the data may include the diameter of the blood vessel, and the shape of the device may be selected to be a cylinder or cylindroid having a diameter which is slightly larger that the diameter of the blood vessel in its relaxed state. Data pertaining to the shape and/or size of the tissue in its relaxed state may be acquired, for example, by imaging.

In some embodiments of the present invention the shaped device has an expandable shape and the method measures the parameters of the shape (e.g., radius) once expanded. Such measurement may be performed by imaging or by a measuring device mounted on the shaped device and configured to communicate with an external system such as a controller of a radiation-emitting system or the like. In other embodiments, the shape is of a pre-defined nature such as a cylindrical shape, and some of its geometrical parameters, such as a diameter of the cylinder, are estimated by measurements of sensors placed on the cylinder (for example 4 non-collinear sensors positions).

The present inventors also contemplate a catheter made to deploy in a helical shape inside the blood vessel, such that its helix is biased against the inner wall of the blood vessel, as shown in FIG. 4A. The helical shape may include a fraction of a helix turn (e.g., half a turn) or it may include one or more helix turns (e.g., at least two turns). For blood vessels with smaller diameter, the overall length of the helical shape is optionally longer than for blood vessels with larger diameter. This may be achieved by providing the helical shape with a larger pitch and/or larger number of rounds. When the target tissue is the prostate or part thereof, the shaped device may be aligned to a typical urethra geometry, or to a urethra geometry that is specific to the subject.

The fixation may be applied to part of the tissue, leaving other parts of the tissue not fixated. For example, in the case of a helical shaped fixation, the internal blood vessel wall touching the helix may be fixated to the helical shape, while the opposite side to the helix of the vessel may be non-fixated.

The method continues to 12 at which radiation is focused on the fixated tissue so as to modulate the tissue.

The term “modulating” as used herein, refers to a change in the biological function or activity of the tissue, including, without limitation, proliferation, secretion, adhesion, apoptosis, cell-to-cell signaling, and the like. In some embodiments, the modulation at least partially damages the tissue, so as to abrogate, inhibit (partially or completely), slow and/or reverse the progression of a condition. In some embodiments of the present invention the modulation is made to alter a measurable condition. In some embodiments of the present invention the modulation comprises denervation. In some embodiments of the present invention the modulation includes modulating prostate tissue at a pre-planned distance from an urethra, e.g., for treating BPH or the like. In some embodiments of the present invention the modulation includes treatment of atrial fibrillation of the heart by modulating the pulmonary vein entrance to the heart. Other modulations are not excluded from the scope of the present invention.

As used herein, “denervation” refers to the modulation of a nerve so as to induce partial ablation, complete ablation or paralysis of that nerve.

The radiation focused at 12 is optionally and preferably performed using a radiation-emitting system which is non-local with respect to the shaped device. For example, the radiation-emitting system may be a non-invasive radiation-emitting system which is located outside the body. Also contemplated, are embodiments in which the radiation-emitting system is a minimally-invasive radiation-emitting system introduced into an organ other than the organ hosting the shaped device. For example, the shaped device may be introduced to one blood vessel and the radiation-emitting system may be introduced into another blood vessel. Another example is a configuration in which the shaped device is introduced to a blood vessel and the radiation-emitting system is introduced into the esophagus. An additional example is a configuration in which the shaped device is introduced into the urethra and the radiation-emitting system is introduced into the rectum.

Representative examples of radiation types suitable for the present embodiments include, without limitation, HIFU, X-ray, microwave and radiofrequency (RF). In some embodiments of the present invention the radiation is HIFU.

In some embodiments of the present invention the method continues to 13 at which the focused radiation is scanned along a predetermined path corresponding to the shape of the device so as to from a modulation pattern on the tissue. The scanning may be done by moving the radiation-emitting system and/or by diverting the radiation beam using an arrangement of redirecting elements, such as, but not limited to, mirrors, prisms, diffractive elements and the like. Also contemplated is the use of phased array elements. In these embodiments, the scanning is effected by altering the relative phase of the phased array elements.

In various exemplary embodiments of the invention the scanning is performed automatically, e.g., using a controller, based on the predetermined path.

The predetermined path may have any shape. In some embodiments of the present invention the predetermined path forms a non closed loop spanning over 360 degrees about a longitudinal axis. A repetitive example is a helix. A helix is particularly useful when the shape of the fixated tissue is elongated and it is desired to modulate the tissue from all sides. The parameters of the path are optionally and preferably based on the shape of the device to which the tissue is fixated. The number of these parameters is preferably sufficient to define the shape and size of the device. For example, when the shape is a cylinder, the parameters may include diameter and length; when the shape is helical, the parameters may include diameter, pitch and number of turns, etc.

When the method measures the geometrical parameters of the shaped device, once expanded, the size of the path is preferably selected also based on this measurement.

In some embodiments of the present invention, the geometry of the path is selected based on one or more parameters, other than the shape of the tissue. Representative examples of such parameters include, without limitation, the BMI of the patient, the required blood pressure decrease, the age of the patient, the gender of the patient, the weight of the patient, the blood pressure, the insulin absorption level and/or any other parameter of the patient or target tissue. For example, the diameter of the modulation path may be selected based on the BMI, wherein for patients with high BMI the diameter is higher than for patients with low BMI. As a representative example, for a patient with BMI above 30, the treatment path may be at a distance about 2 mm from the inner wall of the blood vessel, for a patient with BMI less than 20, the treatment path may be at a distance about 0.5 mm from the inner wall of the blood vessel, and for other patients, the treatment path may be at a distance above 2 mm and less than 0.5 mm from the inner wall of the blood vessel.

The path may be a continuous path (e.g., a line in three-dimensions along which the focal region of the radiation moves), or it may be a discrete path (e.g., a set of points on the target tissue which are sequentially visited by the focal region). The path may also be selected such that the focal region moves over a surface or a volume. The treatment of multiple points along the path may be sequential, simultaneous, or any combination thereof.

In some embodiments, the path includes multiple treatment points in closed proximity thereamongst such that the treatment at these multiple treatment points is executed at a single positioning of the shaped device. For example, when the treatment is via RF, multiple treatment RF electrodes are mounted in closed proximity to each other on the shaped device.

The path may enclose the entire organ or part thereof. For example, when the organ is a blood vessel, the path may enclose the entire periphery of the blood vessel, in which case the azimuthal angle φ, describing the path, satisfies 0≦φ≦360°, or only a portion of the periphery in which case φ satisfies φ₁≦φ≦φ₂, where φ₂−φ₁<360°, e.g., φ₂−φ₁=60° or φ₂−φ₁=90° or φ₂−φ₁=120° or φ₂−φ₁=150° or φ₂−φ₁=180° or φ₂−φ₁=210° or φ₂−φ₁=240° or φ₂−φ₁=270°.

In some embodiments the modulation path has a non-closed shape, e.g., a shape other that a closed annular shape, and in some embodiments the modulation along the path is performed intermittently. These embodiments are particularly useful when the organ is a blood vessel and it is desired to maintain its physical strength. For example, when the path has a helical shape, two or more treatment cycles are implemented. In some of these cycles, the tissue is modulated along arc sections of the helix, wherein the arcs are characterized by an azimuthal angle φ which is sufficiently less than 360° (e.g., 10°, 20°, 30°, 40°, 50°, 60°, or any other angle) thereby leaving the complementary arc sections untreated. In other cycles, the modulation may be continued along other arc sections. The arc sections are preferably selected so as not to form a continuous closed path of treatment points along the tissue, thereby preventing the formation of a contour of mechanical weakness along the blood vessel.

In some embodiments of the present invention the treatment path is visualized on a display device thereby allowing the physician to follow the path manually. When the scanning is performed automatically, the visualized path may be used by the physician for control and adjustment purposes.

The method ends at 14.

Before providing a further detailed description of the method according to some embodiments of the present invention, attention will be given to the advantages and potential applications offered thereby.

The method of the present embodiments is useful particularly, but not exclusively, for the denervation of nerves leading to a kidney, such as, but not limited to, a renal nerve.

The renal nerves are aligned at the periphery of the renal arteries and renal veins. A schematic illustration of renal nerves is shown in FIG. 2. Shown in FIG. 2 is a cross section of a blood vessel (artery or vein), having an inner lumen 400 which is occupied by blood (not shown), an inner vessel wall 401 known as the tunica intima, an elastin layer 402, a bulk wall layer 403 known as tunica media, and an outer layer 404 known as tunica adventita. Elastin layer 402 is between inner wall 401 and bulk wall layer 403, and outer layer 404 surrounds bulk wall layer 403. Outer layer 404 is innervated by the sympathetic nerves 405, which surround the blood vessel generally from all sides at a distance of from about 0.5 mm to about 3 mm away from inner wall 401.

Elastine layer 402 grants the blood vessel some of its mechanical flexible capacity. It is generally preferred to modulate the renal nerves, typically a major part thereof, without or with minimal damage to the layers forming the wall of the blood vessel, particularly inner wall 401 and elastin layer 402, so as to prevent, minimize or at least reduce risk of hemorrhage, tearing, or breaking of the blood vessel.

Conventional techniques for the ablation of renal artery nerves are based on intravascular catheter RF modulation. In this procedure, a catheter is inserted to the required spot of ablation in the artery, and RF energy is induced to the tissue from within the artery using electrodes (see, e.g., U.S. Pat. No. 7,756,583, the contents of which are hereby incorporated by reference). The present inventors found that RF ablation for the treatment of renal nerve has several drawbacks. An intravascular catheter based RF signal needs to substantially heat at the point of contact, in order to affect the target nerve, since it is not capable of generating heat other than at touch-point. To reach physiological ablation of a nerve a temperature of about 60° is typically required for a time period of a few seconds to minutes. For such amount of heat to reach the nerve, the temperature at the point of contact between the catheter and the inner wall 401 should be about 70°. This, however, results in damage to the inner layers of the blood vessel, particularly the inner wall 401 and elastin 402. Additionally, due to substantial heat losses, such an approach in limited only to nerves being very close (about 1 mm or less) to outer wall 404, while it is recognized by the present inventors that renal nerves, for example, are located at deeper depths from the arterial wall.

Another conventional technique includes use of intravascular HIFU catheters (see, e.g., U.S. Published Application No. 20110112400, the contents of which are hereby incorporated by reference). In this technique, a HIFU transducer is built at the distal end of a catheter, so as to transmit energy to the renal nerves. The present inventors found that intravascular HIFU for the treatment of renal nerve has several drawbacks. HIFU transmitters can create extensive damage when the energy flux on the contact area of the transducer is too high. Common safety standards limit such a flux not to exceed 3 w/cm2. Therefore, HIFU transducers are limited in the total transmitted energy by the size of the contact area between the transducer and the contact tissue. The renal artery at the area of treatment may be quite narrow, typically no more than 6 to 7 mm in diameter [“Original Research: MDCT Angiography of the Renal Arteries in Patients with Atherosclerotic Renal Artery Stenosis: Implications for Renal Artery Stenting with Distal Protection”, American Journal of Roentgenology, June 2007, Vol. 188:6, pp. 1652-1658], and this substantially limits the amount of energy that an internal transducer can transmit. The size of the blood vessel hence imposes a minimal contact area between the transducer and the inner blood vessel wall (no more than 1 to 2 cm2) and therefore substantially limits the outwardly depth of treatment due to wave attenuation.

The attenuation of ultrasound waves in tissue may be expressed as exp(α₀f^{1.2}x), where α0 is a coefficient, f is the frequency of the wave, and x is the propagation distance. In order to be able to control direction and focus of ultrasonic energy, the components of the internal ultrasound system are typically a few wavelengths in size. According to calculations performed by the present inventors, the required frequency for an internal HIFU system is generally high. For example, for a 7 mm diameter of blood vessel required frequency is typically about 5 MHz or more. High frequency, however, imposes a very short effective distance from the ultrasound source, in particular with a low power transducer. Thus, according to the above calculations the use of intravascular HIFU is generally ineffective.

Another drawback of conventional intravascular HIFU system relates to the risk of burns, for example, when the internal ultrasound transducer is not coupled well to the arterial wall. To avoid such risk conventional intravascular HIFU catheters oftentimes employ an intravascular balloon, so as to ensure good coupling to the vessel wall. This, however, blocks the blood flow in the treated vessel during treatment, thereby limiting the duration of treatment and its effectiveness.

The technique of the present embodiments overcomes the above deficiencies by providing radiation-emitting system positioned away from a tissue target and optionally outside the body. For example, when the radiation is HIFU, it is not bound by a small contact area with the tissue because it is coupled to the skin, and can assure sufficient ultrasonic coupling, e.g., using impedance matching substances and the like. Thus, there is a wide range of possible frequencies and intensities that result in an effective modulation of the nerve. In various exemplary embodiments of the invention the method employs ultrasound at frequency of from about 400 kHz to about 4 MHz.

The technique of the present embodiments is advantageous also over other conventional techniques such as the aforementioned MRI or ultrasound image guided external HIFU, particularly in clinical situations in which the target tissue moves due to breathing, and/or when the required spatial accuracy is higher than providable by the ultrasound or MRI systems. A particular example is the case of renal nerve ablation for which the preferred spatial resolution is in the sub-millimeter range, which is not providable by speckle or spectral analysis. Such resolution is also not providable by standard MRI since the renal nerves move while the subject is breathing, and the long shutter time of standard MRI does not allow acquisition at sufficient spatial resolution of moving objects. While some modern MRI techniques allow the imaging of electron beam, these techniques are costly and technologically difficult to employ. Use of high enough power level to create detectable mechanical artifacts is also not desired, as indicated hereinabove.

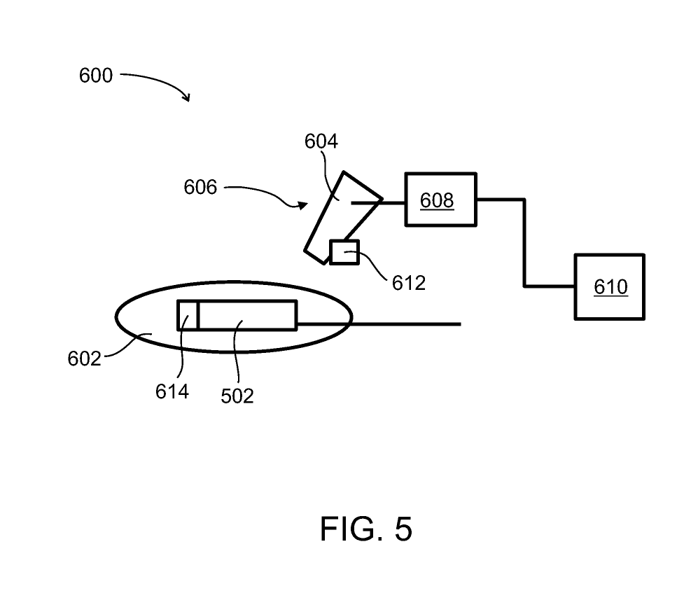

The embodiment of the invention in which scanning is performed along a predetermined path is also advantageous over conventional image guided techniques since conventional techniques typically require a manual control of the beam by the operator, and are therefore susceptible to human error. The employment of predetermined path according to some embodiments of the present invention overcomes this susceptibility since it may be performed automatically. Yet, use of semi-automatic means for controlling the scan along the path is not excluded from the scope of the present invention. For example, the system of the present embodiments may automatically track a movement of a renal artery, allowing the operator to shape the treatment path relative to the artery, without needing to deal with the movement of the artery due to respiration. In these embodiments the system optionally and preferably includes a computer screen in which an image of the artery is displayed in a static position, and the operator shapes the path around it. The system tracks the motion of the artery, and moves the treatment beam relative to the position of the artery according to the operator instructions, compensating automatically for organ movements. In some embodiments a controller (such as controller 604 in FIG. 5) directs an energy beam according to a combination of a) information provided by sensor-based aiming techniques as described with reference to a variety of embodiments presented herein, and b) operator provided information regarding positions of target tissues defined with respect to positions of the sensors. It is this combination that can enable an operator to designate a treatment path (designated relatively to positions of sensor(s) and/or position of a treatment target immobilized with respect to the sensors) on a graphic interface showing a static image, and have the system dynamically aim energy towards the target tissues designated by the operator on the static image, although the system, responding to target designations and/or other commands presented on the static image, is in fact engaging in real-time tracking of and aiming toward a moving target.

Since the tissue according to some embodiments of the present invention is fixated, the path may be set in advanced and be programmed into the scanning system, thus reducing risk of damaging tissues nearby the target tissue. Thus, instead of shaping the path of the treatment beam to the shape of the target tissue, the present embodiments take an opposite approach. The target tissue is shaped to a geometry in accordance with a preconfigured required shape. In some embodiments of the present invention the preconfigured shape of tissue is aligned with a preconfigured treatment path, or treatment points of the radiation beam. In some embodiments of the present invention, the path shape is defined parametrically, wherein during the procedure, shape parameters that are specific to the patient and organ are collected to determine the exact treatment locations.

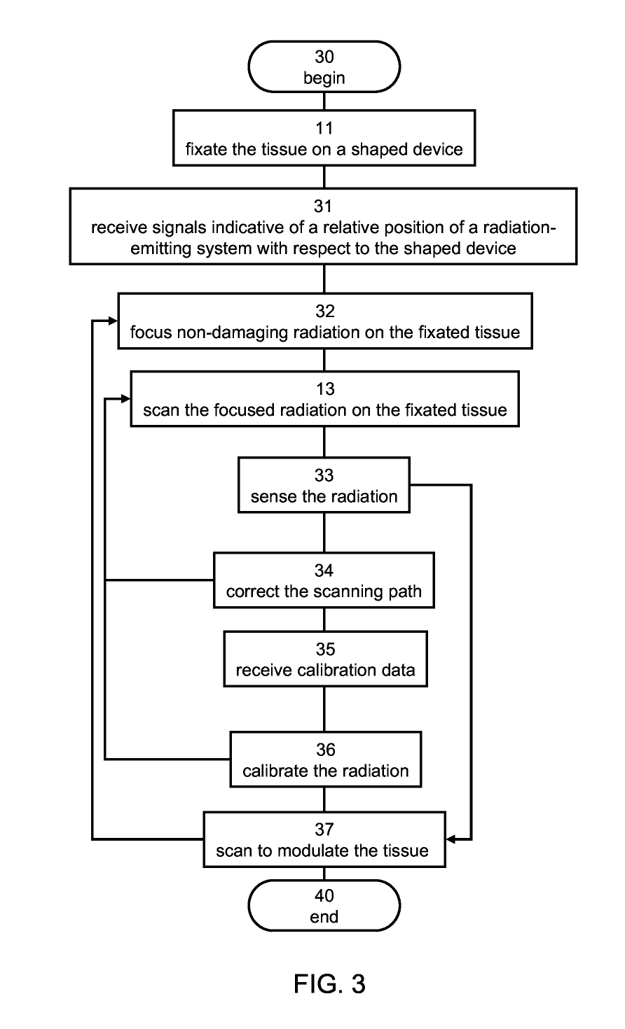

Reference is now made to FIG. 3, which is a flowchart diagram describing in greater detail a method according to some embodiments of the present invention, and to FIG. 4A, which is a schematic illustration of a catheter system, according to some embodiments of the present invention.

In FIG. 3, the method begins at 30 and continues to 11 at which the tissue is fixated as further detailed hereinabove. A representative example of the fixating procedure is illustrated in FIG. 4A which schematically illustrates a catheter system 500 having a shaped device 502 which may be mounted, for example, at a distal end 506 of a catheter 508, according to some embodiments of the present invention. In this representative example, shaped device 502 has a helical shape, which is particularly useful for fixating the internal wall 401 of a blood vessel such as, but not limited to, a renal artery. However, this need not necessarily be the case, since, for some applications, it may be desired to have a shaped device having a shape other than helical as indicated above.

The size of device 502 and catheter 508 is selected in accordance of the size of the artery to be treated. Optionally and preferably, multiple devices of different shapes and sizes are provided to allow the operator to select the most suitable device for the procedure.

The method optionally continues to 31 at which signals indicative of a relative position of the radiation-emitting system with respect to the shaped device are received. This may be achieved, for example, using one or more sensors 504a, 504b and 504c mounted on device 502 and/or catheter 508 and configured for sensing the position of the shaped device 502 within a living body (e.g., within a lumen 400 of a blood vessel) and for transmitting signals pertaining to this position. Optionally, sensors 504 may be energy sensors able to detect energy radiated by the radiation-emitting system. Optionally, the plurality of sensors 504 are not co-planar. It is noted that there is no need for a position sensor reporting an absolute position of the shaped device, because the system of the present embodiments is configured to direct the beam at, or at a known position with respect to, sensors 504. This embodiments is advantageous over conventional system which do not employ fixation. Some embodiments employ a shaped device which is immobilized with respect to tissue by friction, pressure, or another method of attachment. Some embodiments employ a shaped device, such as for example an expandable shape device, which expands to assume a shape similar to that of an existing tissue, thereby optionally immobilizing one with respect to the other. (This may be contrasted to a use of sensors in prior art, where position sensors attached to a catheter tips allows determining the relative position of the tip with respect to the treatment system, but not with respect to the position of the target tissue itself.) This is because the offset between the tip and the target tissue is unknown, both in magnitude and in direction. Piezoelectric sensors, or PVDF sensors, or electro-optic sensors, or temperature sensors, or x-ray sensors, are a partial and exemplary list of types of sensors which might be used.

Optionally, the geometrical setting of the sensors 504 near a target site may be such that a target treatment path, relative to all or some of the sensors positions, is well defined. For example, in treating renal artery surrounded by nerves, sensors placed at inside the artery and touching the artery wall can have a predictable spatial relationship with positions of nerves whose denervation is desired. Therefore, using the sensors, a treatment path (e.g. the path of a denervating energy beam) can be defined around the artery by defining it with respect to sensor positions within the artery. In some embodiments sensors are placed inside an expanding cage in an artery, the sensors placed in a known relation to the cage known geometrical shape (for example two sensors in the axis of the cage, and one at a side touching the artery wall). Other clinical situations fit for such a system are the treatment of atrial fibrillation by pulmonary vein isolation, where sensors are placed at an expanding cage, at the distal end of an intravascular catheter, which is made to fit the entrance of the vein to the atrium, and the shape of the cage made to park at a known position relative to the entry point; sensors are positioned at the cage such that their position is fixed relative to the target tissue, or fixed relative to the shape of treatment (e.g. in the example above, at the arterial wall adjacent to the vein entrance)

The method optionally continues to 32 at which a non-damaging radiation is focused on the fixated tissue.

As used herein “non-damaging radiation” refers to radiation having intensity and duration selected such as not to cause irreversible modulation to the target tissue.

The use of non-damaging radiation is advantageous since it allows to adjust the scanning path and calibrate the radiation parameters (also called the “beam aiming parameters” herein) without causing damage or with minimal damage to non-targeted tissue.

In various exemplary embodiments of the invention the method continues to 13 at which the focused radiation, which some embodiments is non-damaging, is scanned along a predetermined path, as further detailed hereinabove. At 33 the radiation is sensed at or in proximity to the shaped device. This may be done using one or more radiation sensors, configured to respond to the radiation beam. Referring again to FIG. 4A, the present embodiments contemplate a configuration in which one or more of the sensors 504a, 504b and 504c are radiation sensors. In alternative embodiments, some or all of sensors 504 may be position sensors, such as, for example, sensors which detect a position-dependent electromagnetic field generated by a position-detection system.

Thus, in some embodiments a catheter system comprises one or more position sensor(s), optionally without any other type of sensor; in some embodiments a catheter system comprises one or more radiation sensor(s), optionally without any other type of sensor; and in some embodiments a catheter system comprises one or more position sensor(s) as well as one or more radiation sensor(s).

The radiation sensors may be positioned in a known geometrical relationship with the fixation structure of the shaped device. For example, for directing an external HIFU beam, a set of pressure sensors may be placed in known geometry with relation to the fixation structure of the shaped device. The radiation may initially scan the approximate target treatment area, and the beam parameters (e.g., phase shift, amplitude) as sensed by each sensor may be recorded. As a representative example, the beam parameters for which a sensor senses maximum pressure amplitude may be recorded, individually for each of the sensors. Thereafter, one or more such recordings may be correlated with the preplanned path of treatment and/or points of treatment.

In some embodiments of the present invention the position of the focal region relative to the sensor is calculated, for example, using a data processor, based on the signals received from the sensors. Optionally, such calculation is performed without calculating the absolute position of the sensors. From the geometrical relationship between the sensors and the fixation structure, and the information regarding the position of the focal region relative to the sensors, the method optionally and preferably calculates the position of the focal region relative to the fixation structure, hence also the position of the focal region relative to the fixated tissue.

Embodiments in which the absolute position of the sensors is also calculated are not excluded from the scope of the present invention. In these embodiments, the method may receive information pertaining to the location of the fixation structure with the body and uses this information, together with the geometrical relationship between the sensors and the fixation structure, for obtaining the location of the sensors.

In a representative example shown in FIG. 4A, device 502 is helical and comprises three or more radiation sensors, where one sensor (sensor 504a in FIG. 4A) is at the beginning of the helix, one sensor (sensor 504c in FIG. 4A) is at the end of the helix, and one sensor (sensor 504b in FIG. 4A) is approximately at the middle of the helix, optionally and preferably at a position that is not collinear with the other two sensors. Other arrangements and numbers of sensors are not excluded from the scope of the present invention.

The method may thus correct 34 the treatment path (e.g., location, radius) based on signals received from sensors 504a, 504b and 504c such that the treatment path or treatment points follow the shape of device 502 at predefined offset into the tissue. For example, the method may keep the focal region of the focused radiation at a distance of 0.5-3.5 mm outwardly from device 502 so as to assure treating the renal nerves lining the artery with reduced or no damage to the inner wall.

The sensors may communicate with the radiation-emitting system by wire or wireless communication. The present Inventors contemplate many types of sensors and sensor arrangements. In some embodiments, the sensors are arranged at a plurality of discrete locations relative to the shaped device, e.g., as illustrated in FIGS. 4A and 4B, and the sensing is therefore performed selectively at the location of the sensors.

In some embodiments, the radiation is sensed by imaging wherein a data processor executes an image analysis procedure so as to identify focal regions corresponding to the focused radiation. Preferably, the imaging is performed intracorporeally. For example, when the tissue is a blood vessel, a miniature intravascular imaging system is employed. The imaging system may be mounted for example, on the catheter. The imaging system may also be mounted on a trans-esophagus catheter or any other intracorporeal device.

Any type of imaging that can be used for detecting focal regions may be employed. When the radiation is HIFU radiation, the imaging preferably comprises ultrasound imaging, wherein the acquired ultrasound images are then processed to detect focal regions in the image. The focal region may be detected by identifying mechanical vibrations of the tissue in response to the focused radiation, by analyzing speckles in the image, by spectral analysis of the signal, or any other image analysis technique or combination of techniques.

In some embodiments of the present invention, the sensing is by reflecting the radiation outwardly and collecting the reflected radiation outside the body. In these embodiments, the shaped device may be mounted with one or more reflectors which reflect the radiation outwardly. For example, a reflector 505 (examples are labeled 505a, 505b and 505c in the FIG. 4A) may optionally be positioned at or near the location of one or more of sensors 504a, 504b and 504c. The reflectors may replace the sensors or they may be provided in addition to the sensors.

The reflected radiation may be sensed using a dedicated set of sensors arranged outside the body. Such sensors may be arranged, for example, on the radiation-emitting system. Alternatively or additionally, the radiation-emitting system, e.g., HIFU system, may be configured to receive the reflected radiation, e.g., by means of transceivers configured to receive radiation at the wavelength of the reflected radiation. The method may record the radiation parameters for each reflector, for example, when the corresponding reflected radiation is maximal. Use of reflectors is advantageous from the standpoints of cost and availability.

The present embodiments differ from diagnostic systems, such as diagnostic ultrasound, because it is not necessary to extract spatial resolution from the reflected radiation. Specifically, since the position of the reflector is known, only the radiation parameters (amplitude, phase, or other parameters) of the reflected radiation are analyzed. In some embodiments of the present invention the receiver has a narrow band which is adapted for the wavelength of the emitted radiation. This is unlike diagnostic systems, e.g., diagnostic ultrasound in which the bandwidth is made wide to improve signal to noise ratio.

In some embodiments of the present invention reflectors 505 are switchable and the method switches the reflectors on and off so as to associate the reflected radiation with each sensor. For example, a reflector may be made switchable by placing it in a capsule, e.g., within the structure of the catheter, and rotating it, e.g., mechanically or by applying a magnetic field, such that when it points to one direction it is considered in an “on” state and when it points to another direction it is considered in an “off” state.

In some embodiments of the present invention the reflector is encapsulated within a capsule which is fillable with fluid. These embodiments are particularly useful when the applied radiation is ultrasound radiation, whereby when the capsule is filled with liquid, the liquid vibrates with the ultrasound wave. Such an encapsulated sensor may be switch off by introducing gas into the capsule and switched on by introducing liquid into the capsule. Thus, for example, the capsules may be initially filled with liquid and the method may selectively introduce gas into the capsules to evacuate at least a portion of the liquid. Conversely, the capsules may be initially filled with gas and the method may selectively introduce liquid into the capsules evacuate at least a portion of the gas. Also contemplated, are embodiments in which the method repeats the procedure, namely introduce liquid after liquid evacuation and/or gas after gas evacuation. In any of these embodiments the method analyzes the resulting changes in the reflected radiation to associate the reflected radiation with individual capsules.

In some embodiments of the present invention the reflectors modulate the radiation upon reflection wherein different sensors may detect (and/or are selectively sensitive to) different modulations, and the method associates the reflected radiation with each sensor based on the modulation. Such modulation may include switching between sensor states at an identifiable frequency. For example, reflector 505a may be switched on and off periodically as a first rate, reflector 505b may be switched on and off periodically as a second rate, and reflector 505c may be switched on and off periodically as a third rate. The association of reflective radiation with a particular reflector may be achieved by filtering the reflected radiation according to the respective rate. Thus, the modulation encodes spatial information into the reflected radiation.

Once the scanning path is corrected, the method optionally and preferably loops back to 13, so that the operations 13, 33 and 34 are performed iteratively, until a predetermined accuracy level is achieved.

The method then proceeds to 35 at which the method receives calibration data, and to 36 at which the radiation is calibrated based on the received calibration data. The calibration data may be prepared in advance, for example, at laboratory calibration time or the like. A calibration system may move a radiation sensor in three-dimensions to cover all operational volume designed for the treatment. For each position of the sensor, the calibration system may focus the radiation beam onto the sensor, for example, by scanning the region in the neighborhood of the sensor until a desired reading, e.g., maximum amplitude, is obtained from the sensor. For each such position, the corresponding radiation parameters for that position are recorded. Representative examples of such parameters including, without limitation, phase shift of the radiation and intensity amplitude. The resulted data thus includes a set of position values (typically coordinates in three-dimensions) and corresponding radiation parameters. The data may be stored, for example, as a multidimensional matrix, prior to the execution of the medical procedure, or as factory settings.

In various exemplary embodiments of the invention the calibration 36 comprises correlating the calibration data with the radiation parameters at the relative position of the sensor with respect to the fixating structure or tissue. For example, the method may search for the expected relative position of each sensor based on the position values in the calibration data. This expected position typically deviates from the relative position of the sensor due to, e.g., body and environment distortions. The method then employs an interpolation algorithm for calculating the radiation parameters based on the relation between the expected positions from the calibration data and the known relative positions of the sensors.

Once the radiation is calibrated the method optionally and preferably loops back to 13, so that at least some of operations 13, 33, 34 and 36 are performed iteratively until a predetermined calibration accuracy is achieved. In other embodiments, the sensors read one or more transmitter elements, the system determines their parameters (such as, but not limited to, phase and amplitude), and iteratively loops over other elements, each time correlating the information to calibration information obtained in the laboratory, to analyze relative intensities, phases, and other such parameters of each portion of the whole transmitting system.

The method may then proceed to 37 at which the radiation is scanned along the treatment path so as to modulate the tissue. The fixation of tissue to the known geometry, the fixation of the sensors to the same geometry, and the communication between the radiation-emitting system and the sensors according to some embodiments of the present invention allows for an accurate treatment with reduced or no damage to non-target tissue. Optionally, the method may measure the parameters of only a portion of the transmitting elements, and determine the transmission parameters for treatment for all elements by means of algorithms such as interpolation or extrapolation of parameters.

The present Inventors recognize that during treatment, breathing, heartbeat, digestion, patient movement and other movements may alter the geometrical relationship between the beam transmitters and the fixated geometry of the target tissue.

In some embodiments of the present invention, the alignment between the fixated tissue and the treatment path is updated so as to compensate for tissue movement, or any other misalignment that may occur in time during treatment. In some embodiments, once a portion of the tissue is modulated, the method loops back from 37 to 32, the radiation is reduced to non-damaging levels and the method corrects the path based on the sensed radiation and/or calibration data as further detailed hereinabove. The rate and duration at which the path is updated is optionally and preferably predetermined. For example, the method may operate at a period P and duty cycle D, wherein during the period P, the method scan to modulate the tissue for a time-interval P·D, and performs path updates for a time-interval P·(1−D). Typically, but not necessarily, P is from about 10 milliseconds to about 1 second, and D is from about 0.5 to about 0.95. In some embodiments, alignment between fixated tissue and the treatment path is updated whenever movement of the tissue exceeds a predetermined portion of the size of the region of focus of the beam. The duration in which the target tissue movement is bigger than that of a predetermined portion of the focal size may be preconfigured or measured by analyzing the results at 13.

A representative example of the operations during a period P is as follows. The method initially determines the location of the sensors, treatment path and radiation parameters as further detailed hereinabove. The method stores the positions of the sensors and modulates the tissue for the tissue-modulation time-interval P·D. The radiation is then reduced to a non-damaging level for the path-update interval P·(1−D). During the path-update time-interval, the method focuses the radiation onto one of the previously stored locations of the sensors. For each such location, the method moves the focal region at the vicinity of the location, preferably in three-dimensions (e.g., at each of the six directions: front, back, left, right, up and down), to allow the sensor to responsively sense the change in radiation. The method may use interpolation or extrapolation methods for calculating the parameters of transmitters that were not measured during the measurement cycle. The method may use interpolation or extrapolation algorithms to update transmission parameters so as to adopt preferable treatment positions with respect to target tissue movement in between measurement cycles.

For example, when the radiation in HIFU, the sensor may be a pressure sensor which senses changes in the pressure amplitude when the HIFU focal region moves at the vicinity of the sensor. Once the sensed amplitude reaches a local maximum, the method determines that the HIFU is directed onto the sensor and stores the updated location or direction, and optionally also other radiation parameters. The updated information is then used for updating the treatment path. It is to be understood that this procedure may also be used for other sensed parameters and other types of radiation as indicated above.

The radiation level is then increased back to the damaging level and a new cycle is executed wherein the tissue is modulated along the updated path. The above procedure is optionally and preferably executed for each of the sensors. In some embodiments of the present invention locations of two or more (e.g., all) the sensors are updated during a single path-update time-interval, and in some embodiments of the present invention the locations are updated intermittently, namely the locations of at least two sensors are updated during different cycles.

In some embodiments of the present invention, signals received during consecutive measurements, either in the same cycle or consecutive cycles, are analyzed so as to determine the motion vector and optionally also the acceleration of the focus relative to the shaped device. The motion vector may then be used to calculate, typically by a data processor, the path of focal region, to compare the calculated path with the treatment path, and to update the scan based on the comparison. The scanning is optionally performed continuously along the preconfigured treatment path. Performing the scan in a continuous manner is advantageous, because it reduces the probability of damage to non-target tissue without decreasing the effectiveness of the treatment. In some embodiments, estimated path parameters such as position, velocity, and acceleration of each sensor are calculated, and during this calculation, the system updates the position of the treatment beam according to previously calculated parameters

Also contemplated, are embodiments in which the radiation is sensed also during the tissue-modulation time-interval. The method may update the path or intensity or any other transmission parameter during treatment, namely without reducing the radiation level of each of the sensors during treatment, or, more preferably, the method may reduce the radiation once the method determines that the difference between the sensed radiation and the expected radiation, for a particular sensor, is above a predetermined thresholds. Thus, in these embodiments, the method loops back from 37 to 33 at which the radiation is sensed. If the difference between the sensed and expected radiation is below the threshold, the method returns directly to 37. If the difference between the sensed and expected radiation is not below the threshold, the method loops back to 32, the radiation is reduced to a non-damaging level and a cycle or period P is initiated as further detailed hereinabove. For clarity of presentation, transitions corresponding to these embodiment (e.g., from 37 to 33, and from 33 to 32) are not shown in FIG. 4A, but one of ordinary skills in the art, provided with the details described herein would know how to adjust the flowchart to show such transitions.

The method ends at 40.

Reference is now made to FIG. 4B, which is a schematic illustration of a shaped device according to some embodiments of the present invention. Shaped device 502 is optionally an expandable shaped device 512, as shown in the figure. The embodiment shown in FIG. 4B is designed to adopt a contracted profile during insertion, for example by being constrained to a narrow profile by being contained in a guiding sheath (not shown), and to adopt an expanded profile (as shown in the figure) when the expandable portion of the device is extended beyond a distal end of the constraining guiding sheath, and/or when the guiding sheath is retracted from around the expandable portion. As mentioned above, similar devices may be made to assume an expanded configuration in response to mechanical, thermal and/or electrical activation. In an optional method of use, expandable shaped device 512 is inserted into a body lumen, such as for example a blood vessel or an esophagus, and there caused to expand. The expanded device 512 may be expanded until its expandable arms 519 come in contact with, and optionally slightly stretch, walls of the lumen in which it has been inserted. Several purposes may be accomplished. One is to immobilize device 512 within the lumen into which it is inserted, so that sensors carried by device 512 come to have a fixed position with respect to the lumen and whatever is connected to walls of the lumen. Another is to enable manipulation of the position of the lumen walls and whatever is connected to them by manipulating the position of device 512, for example to move an organ into which device 512 out of danger while a nearby organ is being treated. Yet another is to cause the lumen walls to assume a pre-planned shape, for example to facilitate treatment of the walls and/or associated tissues. Yet another is to calculate target tissue position relative to sensors mounted on the device, positioned in a shape known to correspond to a shape of the tissues.

It is to be understood that the specific designs shown in FIGS. 4A and 4B are exemplary only, and not to be considered limiting. In particular with respect to FIG. 4B, it is noted that any expandable cage-like and/or mesh-like expandable structure may be used, for example, to fix a surrounding blood vessel to a pre-determined shape, for example to a shape which is cylindrical, or to a shape which is squared with rounded corners, and which is optionally approximately co-axial to the catheter.

Optionally, expandable shaped device 512 may comprise one or more sensors 504 and/or reflectors 505, as discussed hereinabove and as shown in FIG. 4B.

Reference is now made to FIG. 5 which is a schematic illustration of a system 600 for modulating tissue of an internal organ in vivo. System 600 comprises one or more shaped devices adapted for being introduced into a living body 602 and being configured for fixating a tissue thereon so as to shape the tissue generally according to a shape of the device. One or more of the shaped devices may be for example, device 502, in which case system 600 preferably comprises catheter system 500 including shaped device 502, catheter 508 and optionally also sensors 504a-c and/or reflectors 505a-c, as further detailed hereinabove.

While FIG. 5 shows a system including a single shaped device, it is not intended to limit the scope of the present invention to such configuration. For example, the system may comprise a plurality of shaped devices, whereby different devices are adapted for being introduced to different locations in the body. For example, different devices may be adapted for being introduced into different blood vessels. In some embodiments of the present invention different devices are adapted for being introduced into different locations in the same blood vessel.

System 600 further comprises a radiation-emitting system 604 configured for emitting radiation from a location 606 external to body 602 and focusing the radiation on the fixated tissue. In various exemplary embodiments of the invention system 600 comprises a scanning system 608 operative to scan the radiation over the fixated tissue. Scanning system 608 may move radiation-emitting system 604 and/or it may divert the radiation using an arrangement of redirecting elements (not shown), such as, but not limited to, mirrors, prisms, diffractive elements and the like. In various exemplary embodiments of the invention the scanning is performed automatically, e.g., using a controller 610 configured for controlling radiation-emitting system 604 and scanning system 608 such that the scan is along a predetermined path corresponding to the shape of device 502. Controller 610 may include or be supplemented with a data processor configured for performing the calculations described herein, and scanning system 608 may optionally be part of controller 610.

It is noted that in some embodiments the functionality of scanning system 608 is included within functions of controller 610, while in other embodiments (where scanning system 608 is a mechanical device, for example), beam aiming operations may require participation of emitter 604 and also of scanning device 608, both under control of controller 610. For simplicity of exposition, in this document references to beam controlling operations effected by controller 610 should be understood as including, in some embodiments, operations in which scanning device 608, directed by controller 610, participates as well.

When the system comprises a plurality of shaped devices scanning system 608 scans the tissues fixated on at least some of each of the shaped devices, more preferably on each of the shaped devices. Optionally, each such tissue is treated separately, so as to reduce the risk of damaging neighboring tissue. Alternatively, a path may be selected to allow treating two or more tissues that are fixated on different devices during the same scan. For example, the path may have the shape of a figure of 8 around and between two blood vessels (e.g., an artery and a vein). Optionally, such path is preplanned. Optionally, the path is automatically selected based on the geometry of the fixated tissues and shaped devices. Optionally, the path is manually chosen by an operator, and the system uses the sensor device for omitting selected organs (e.g., vital organs such as, but not limited to, artery, vein, urethra) that are not to be affected, and/or for compensating for organ movement.

When device 502 comprises one or more sensors (e.g. sensors 504, shown in FIG. 4A and FIG. 4B) for sensing the radiation or position of the shaped device, controller 610 receives signals from the sensor(s) and controls radiation-emitting system 604 and scanning system 608, responsively to the received signals, as discussed in further detail hereinbelow.

When device 502 comprises one or more reflectors 505 for reflecting the emitted radiation, system 600 may comprise one or more radiation sensors or receivers 612 configured for sensing the reflected radiation at one or more sensing locations external to the body, as further detailed hereinabove. In these embodiments, controller 610 receives the signals from the radiation sensor 612 or receiver and controls radiation-emitting system 604 and scanning system 608, responsively to the received signals.

In various exemplary embodiments of the invention controller 610 is configured for calibrating the radiation. In some embodiments of the present invention the calibration is performed in response to the signals received from the sensors. In some embodiments of the present invention controller 610 accesses prerecorded calibration data having a plurality of entries, each entry comprising a set of radiation parameters associated with a three-dimensional coordinate. Controller 610 searches the data for a 3D coordinate corresponding to a sensing location and extracts a respective set of radiation parameters. Controller 610 then calibrates system 604 based on the respective set of radiation parameters.

In some embodiments, system 600 comprising an intracorporeal imaging system 614 configured for imaging the fixated tissue and regions in proximity thereto. In these embodiments, controller 610 analyzes imagery data received from intracorporeal imaging system 614, and identifies focal regions corresponding to the focused radiation, as further detailed hereinabove. In an exemplary embodiment shown in FIG. 5, intracorporeal imaging system 614 is shown as being contiguous to device 502, yet this configuration is exemplary and not limiting. Imaging system 614 might, for example, be positioned in a first organ and imaging system 614 in a nearby second organ.

The present embodiments also contemplate configurations in which the treatment system 604 is intracorporeal, and configurations in which both intracorporeal and an extracorporeal systems are employed. In some embodiments, device 502 aligns a treatment tool, such as an electrode, an intravascular HIFU reflector or transducer, or any other mechanism that causes treatment, to move along the predetermined path within the body 602. For example, device 502 may include an RF electrode movable along the catheter 508. Alternatively, a set of treatment tools, such as RF electrodes are fixed on the length of the fixation structure such that each affects a location along the shape of device 502. Also contemplated are embodiments in which an intravascular HIFU system (not shown) with a beam of substantially less than 360°, is mounted on device 502 or catheter 508. The intravascular HIFU system may scan the radiation along the predetermined path, e.g., by a reflector or by moving the transducer, so that the focal region moves along the path or along the shape of device 502.

In some embodiments of the present invention multiple devices are positioned in organs in the vicinity of target tissue for enabling treatment path of a treatment beam to be conducted safely and without harming non-target tissue. For example, in some embodiments for treating atrial fibrillation, a system includes one or more catheters inserted into the initial portion of a pulmonary vein, and a trans-esophageal device inserted into an esophagus. During treatment, the system computes the path of the treatment beam in a way that will not excessively heat the esophagus of the patient. The system may additionally alert the operator for danger of over-heating the esophagus if it identifies that treatment path will hit the esophagus, and may suggest altering the geometrical configuration of the esophagus by moving it, or by altering the patient position. Other examples include a system of multiple catheters for renal denervation, one inserted into the artery, and the other into the vein; once treatment is operated, the system processor assures the path of beam is optimal in energy levels to not harm vein or artery, while conducting a path similar for example to the figure “8”.

In some embodiments of the invention, a system comprises multiple energy transmitters. During treatment, the system conducts repeated cycles of measurement and treatment as described above. In some clinical situations, some of the transmitters' beams are obstructed from reaching target tissue during a portion of the treatment, or during all of it. The system uses sensor readings made during the measurement cycles to determine whether transmissions from each transmitting element can reach the target, and temporarily shuts down the ineffective elements during associated treatment cycles. In this way the system processor can accurately determine the energy dose being delivered to the target tissues, and can calibrate energy levels of transmissions and duration of transmissions accordingly. Additionally, in this way obstructing organs are not affected by unwanted beam portions that might hit and damage them.

In some embodiments of the present invention a projector. Examples are a phased array or annular array elements of a HIFU transmitter. The ultrasound energy which the transmitters emit travels in the speed of sound in the tissue, and therefore each element transmission takes time to reach the sensors. The system in this embodiment optionally measures each element's transmission concurrently at all sensors to optimize measurement cycle duration. In some embodiments, the system iterates the transmissions in a manner that allows transmitting from consecutive elements without waiting for the previous element transmission to reach the sensors; this can be achieved by choosing the next element to transmit to be close by to the previous one, thereby ensuring the sensing order of the beams will be similar to the transmission order.

In some embodiments of the present invention, the system uses measurements of delay between transmissions to multiple sensors with relatively known geometric relationship on the device. The system then calculates required delay of transmission to target tissue, optionally without determining the location of sensor, or target tissue. This method takes into account beam aberrations that may behave differently from one transmitting element to another. By measuring the delay from multiple sensors, the system calculates the required delay for a nearby target position, for each transmitting element—thereby synchronizing all elements to hit target in concert. This method is advantageous, as it does not require imaging of target or beam, and does not require any measurement of location of sensors, target tissue or beam, yet enables conducting accurate beam treatment.

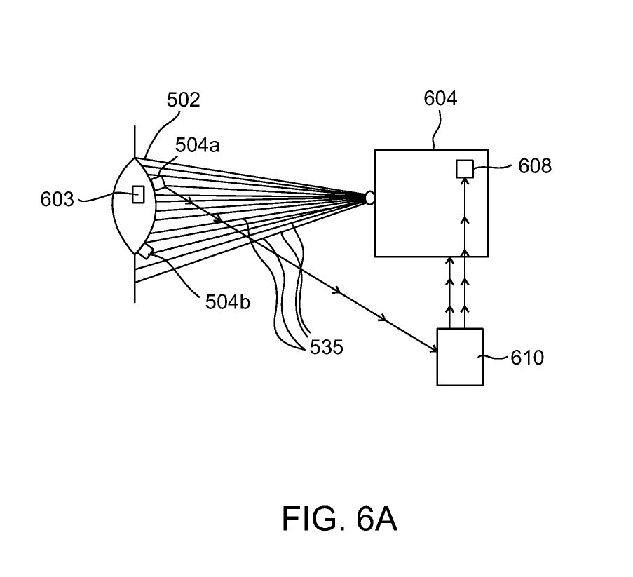

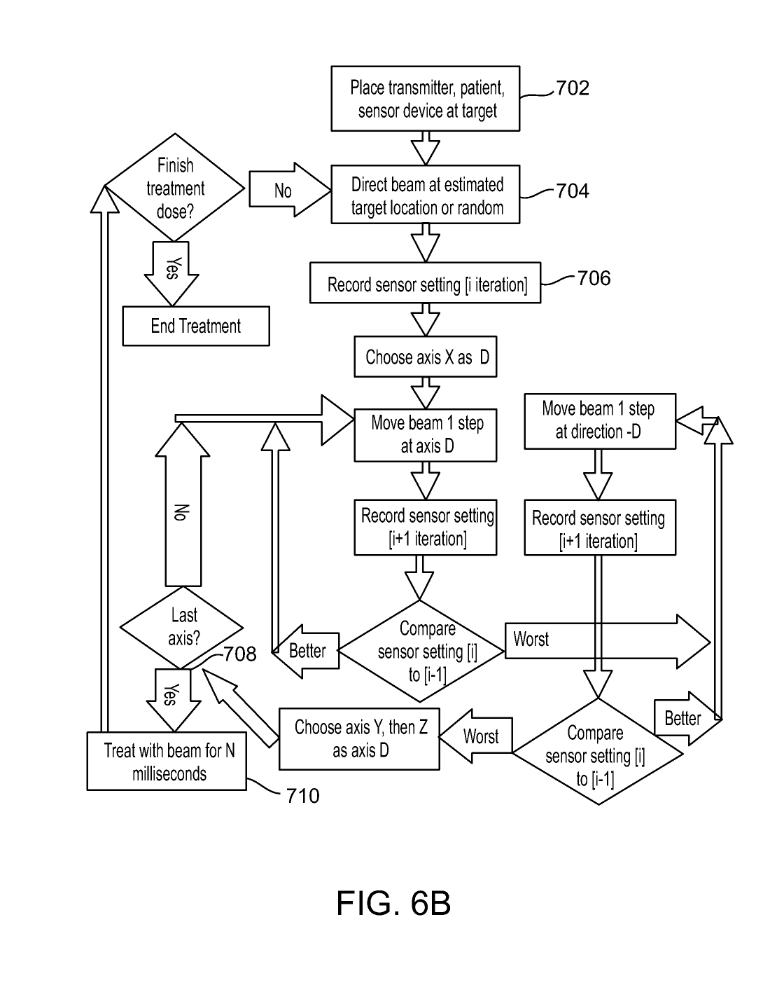

Attention is now drawn to FIG. 6A, which is a schematic illustration of a system for aiming an energy beam with reference to a sensor positioned within a body and near a target tissue, according to some embodiments of the present invention.

FIG. 6A shows radiation emitter 604 emitting radiation generally in the direction of a target tissue 603 having a known or predicted spatial relationship to a sensor 504. Sensor 504 is optionally mounted on a catheter, or on a shaped device 502 optionally mounted on or connected to or delivered by a catheter.

The present invention, in some embodiments thereof, uses a sensor or plurality of sensors positioned near or at target tissue to enable precise directing of beam position relative to target. Sensors 504 are selected to be of a type which senses energy projected by radiation emitter 604. For example, if emitter 604 emits ultrasound, sensor 504 may be a pressure sensor, and if emitter 604 emits x-rays, sensor 504 may be an x-ray sensor. Alternatively and or additionally, sensors 504 may be of type which senses an effect produced by energy projected by radiation emitter 604. For example, if emitter 604 emits ultrasound, sensor 504 may be NIR sensor or temperature sensor.

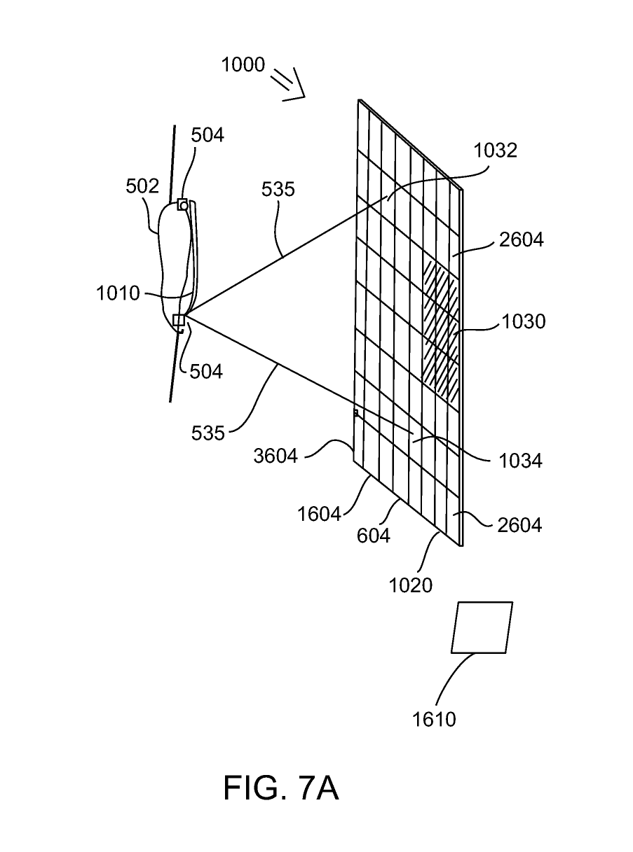

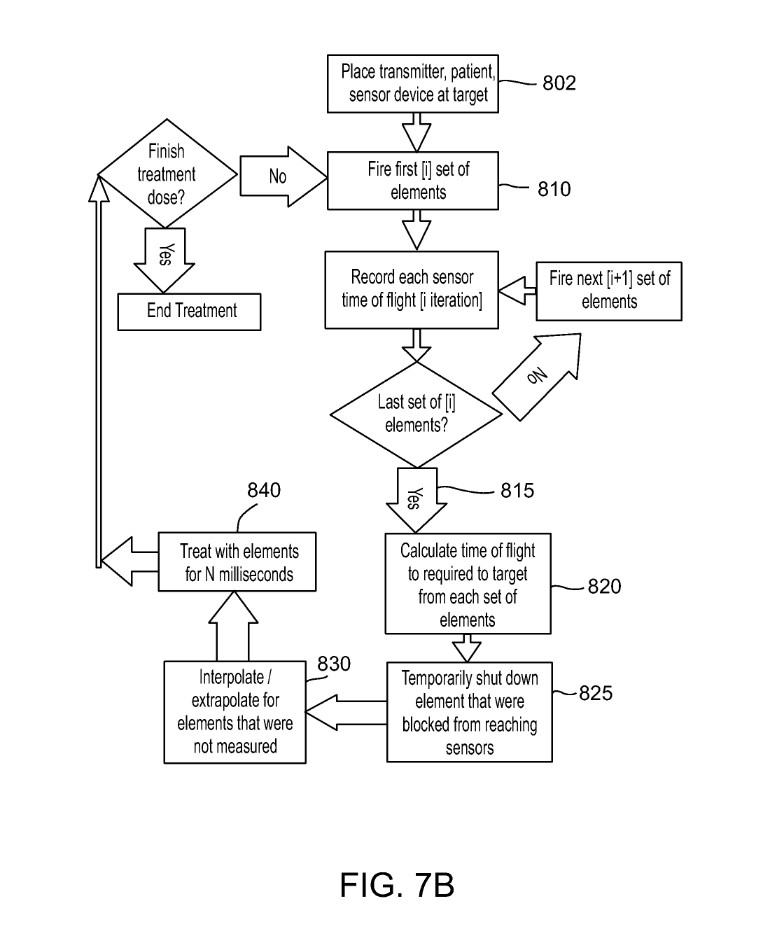

Systems shown in FIGS. 5, 6A and 7A, and methods shown in FIGS. 6B and 7B, discussed below, show methods for using output from sensors 504 to calculate energy beam aiming parameters which direct an energy beam towards sensors 504, and/or towards targets having a known spatial relationship to the sensors 504.

One way to direct the beam to the sensor is to initially direct it at a position in the general direction of the sensor, or even at a random position, and then to iteratively change the beam aiming parameters (i.e. radiation parameters which influence aiming of the radiation) so as to maneuver the beam aim back and forth at each axis in space, and detect amplitude and/or phase and/or timing and/or any other characteristics which characterize energy received at the sensors for each set of beam aiming parameters, and thereby detect which beam aiming parameters maximize intensity of energy received at the sensors and/or maximize some other required characteristic of energy detected by the sensor. FIG. 6A shows a controller 610, optionally utilizing a scanning system 608, sending a plurality of (optionally non-destructive) energy beams 535 in the general direction of a sensor 504. Controller may directly control emitter 604 and/or may control scanning system 608, which directs energy from emitter 604.

For each beam, sensor 504 reports characteristics (e.g. phase and/or amplitude and/or timing and/or other characteristics) of energy detected at sensor 504 following sending of an energy beam 535 by emitter 604. In some embodiments, controller 610 and/or scanning system 608 iteratively changes beam aiming parameters used by emitter 604 to project beams 535.

Controller 610, by comparing readings from sensor 504 for each beam 535, identifies which beam 535, and which associated beam aiming parameters used to send the selected beam, maximizes (or alternatively, minimizes, or alternatively produces a satisfactory level of) energy intensity or some other characteristic of energy received at sensor 504. In this manner, controller 610 discovers what beam aiming parameters used by emitter 604 and/or scanner 608 will effectively aim an energy beam 535 towards sensor 504.

By iterating this process, controller 610 can discover aiming parameters for aiming a beam towards locations of one or more sensors 504. Relevant energy characterizations reported by sensors 504 and/or calculated by controller 610 based on information from sensors 504 may include phase, time of flight (i.e. the delay between beam projection and beam sensing at the sensor), amplitude, and/or any other factor that affects beam displacement and/or characterizes energy detected by the sensor.

Any scanning pattern may be used. For example, in some exemplary embodiments controller 610 may first move a beam in an certain direction on an (optionally arbitrary) X axis, and then if a sensor 504 detects a reduction in beam energy received at the sensor, controller 610 may move the beam in an opposite direction on the X axis. Progressive iterations of this process will determine a set of beam aiming parameters which maximizes energy received at the sensor, over all possible positions of the beam projection on the X axis. This iterative stepping may be reiterated on a Y axis, and then again on a Z axis, or vice versa. Using this technique, the beam parameters such as direction, phase setup, focal distance, and the like which maximize energy received at each sensor may be found. Additional parameters and characteristics of the beam, such as a ratio of received intensity to projected energy may also be recorded; such parameters and characteristics may be used during a treatment phase of operation (defined below), for example to determine projected intensity, or duration, or other parameters required for dose control, safety, or any other requirement of the treatment.

Note that it is to be understood that descriptions herein relating to scanning an energy beam in terms of X, Y, Z coordinates of a Cartesian coordinate system are exemplary only, and not limiting. Similar calculations may be made and similar scanning methods accomplished in terms of an angular coordinate system, or any other coordinate system or method of calculating beam aiming and target positions.

The operation described so far with reference to FIG. 6A is comprised in what is referred to herein and in the attached claims as a “measurement phase of operation”, during which a system such as system 600 learns how to aim energy beams at sensors 504 and/or in directions calculated with reference to positions of sensors 504. Optionally, this measurement phase of operation uses beam energy levels sufficiently strong to be detected by sensors 504, but sufficiently weak to have little or no harmful effects on body tissues. In some embodiments, a measurement phase of operation is followed by a “treatment phase of operation”, wherein emitter 604 (optionally using scanner 608) directs an energy beam strong enough to modulate tissue towards a target tissue using aiming parameters selected by controller 610 during a measurement phase of operation.

The directions of X, Y, and Z, mentioned above may be relative to emitter 604 the transducer, or may be arbitrary.

Procedures described above with reference to FIG. 6A and shown in the figure with reference to a single sensor 504 can be repeated in three dimensions and with respect to a plurality of sensors 504. In some embodiments of the invention a plurality of sensors 504 are used. Optionally these sensors are not all positioned on a same plane. Optionally, the sensors have a known spatial relationship to each other (for example, because they are mounted at different points of a same shaped device 502). In some embodiments the scanning method described with reference to FIG. 6A is used by controller 610 to determine and/or calculated beam projection parameters which will direct an energy beam towards each of the sensors 504. (Measurements may be done for each sensor separately, or by both simultaneously, or a combination of both.) Optionally, controller 610 then uses that information to calculate (for example by interpolation and/or extrapolation) parameters useable for directing an energy beam to a predetermined position with respect to the sensors. If a position of a tissue target relative to sensors 504 is known or predicted, such calculated parameters enable to aim the energy beam at the tissue target and/or at a pattern optionally preplanned to be aimed at a tissue target, such as for example a target defined with respect to the position of a shaped device 502 presumed to have a known positional relationship with respect to a tissue target.

In some embodiments, controller 610 then uses those calculated parameters to aim an energy beam towards a target tissue. In some embodiments a beam so directed has a higher energy than beams used in the measurement phase of operation. Typically, in a treatment phase of operation a beam 535 has sufficient energy to modulate a target tissue.

In some embodiments sensors 504 are positioned at two extremes of a target tissue, and controller 610, using information calculated as shown above, calculates parameters required to direct energy between the sensors and towards the target tissue. In another example, a plurality of sensors 504 are mounted on an expandable catheter component such as expandable shaped device 512 of FIG. 4B. In some embodiments a structure such as shape 512 is expanded within a renal artery, fixing the artery tissue around the expanding structure. In other embodiments a structure such as shape 512 is expanded within a renal artery, taking the general (cylindrical) shape of the artery and optionally fixing to a certain location in the artery during treatment, that is, immobilizing shape 512 and artery portion one with respect to the other (though not necessarily immobilizing them in absolute space. The calculation method described above may then be used to direct energy to points at a predetermined distance around the device 512, thereby directing the energy towards positions where the energy may modulate renal nervous tissue positioned outside the renal artery. In a similar embodiment, expandable device 512, or another shaped device 502, may be positioned and fixed within, for example, a renal artery at a known distance distal or proximal to a target tissue on or adjacent to the artery, and energy may be directed towards that target tissue using methods described above. This embodiment is advantageous in that it removes the sensors 504 from the target area, thereby avoiding danger of damage to the sensors. Optionally, a system for conducting such a treatment may include a guide wire 521 extending distally from a catheter having an open cage structure such as that of device 512 of FIG. 4B. The cage may be positioned with its distal end proximal to the treatment target area, the guide wire ensuring a relative straight path between the cage and a target area distal to the cage along the artery. Guide wire 521 may then be retracted, and a beam treatment effected at a pre-determined length forward of the cage, at a position where the artery is empty of devices. This method is preferable in situations where the shape of ablation is known relative to the position of the sensors, such as renal denervation, and eliminates the need for an imaging system to view either tissues, or beam, as the target treatment path is defined relative to the position of the sensors (in the case of renal denervation, a predetermined shape around the renal artery), and the beam parameters for moving around the sensors may be easily calculated relative to the parameters of the beam recorded at the sensors.

In some renal nerve modulation and in various other situations, target tissue may be expected to move during treatment, because of respiration of the patient, heartbeat, digestive system movements, patient movement during treatment, or for other reasons. In these situations, a useful method for modifying beam position according to tissue movement would include dividing the energy transmission into repeating cycles of activity, and further dividing each cycle into
a first duration during which controller 610 detects and/or calculates aiming parameters which controller 610 and/or optional scanning device 608 and/or emitter 604 can use to direct a beam towards sensors 504 or toward a position related to positions of sensor 504 (this is the measurement phase of operation), and
a second duration, the treatment phase of operation, during which controller 610 and/or optional scanning device 608 and/or emitter 604 use those calculated parameters to direct tissue-modulating energies towards a target tissue.

The duration of a single cycle, and the division of each cycle between measurement phase and treatment phase, depend on the moving characteristics of the tissue to be treated. During renal denervation treatment, for example, a renal artery may move as much as 3 cm during each respiration cycle. In some embodiments a 100 to 200 milliseconds cycle duration is used, and of that time, between 10% and 30% is used for measuring beam position with respect to the sensor or sensors, the rest of the cycle time being available for treating target tissues. In some alternative embodiments, between 1% and 30% are so used. In some embodiments, between 0.1% and 30% are so used.

In some embodiments, controller 610 begins a beam scanning operation of the measurement phase (as described above) at positions recorded as optimal for delivering energy to a sensor during the previous cycle. Additionally or alternatively, controller 610 may optionally be programmed to use algorithms which detect or calculate movement trends, use that information to calculate a predicted next position for each sensor for a next cycle, and start sensor maxima detection from that predicted next sensor position. In some embodiments, controller 610 may optionally be programmed to use algorithms which predict movement of target tissue, to better align the energy beam treatment path to detected and/or predicted movement.

Attention is now drawn to FIG. 6B, which is a simplified flowchart providing additional details of a method of aiming energy towards a target tissue, according to some embodiments of the present invention.

The method of FIG. 6B, in some embodiments thereof, can be roughly summarized as follows:

A tissue-modulating energy beam from a source distant from a target tissue is directed towards that target tissue to treat it. The method comprises
positioning within a body and near a target tissue at least one sensor operable to detect energy from the energy source;
aiming towards a plurality of positions within an intrabody volume containing the sensor a plurality of experimental energy beams of non-damaging intensity, each beam being aimed according to at least one experimental beam-aiming parameter used to aim that beam;
selecting, from among said plurality of experimental beam-aiming parameters, a parameter shown by detection of beam energy at the sensor to have successfully aimed its associated experimental energy beam towards the sensor; and
aiming a tissue-modulating treatment energy beam from the source toward the target tissue according to a treatment-beam-aiming parameter calculated as a function of the selected experimental beam-aiming parameter.

Optionally, the selected beam-aiming parameter is that parameter which, when used to aim a beam, produced maximum energy (from among all the experimental beams) as measured by the sensor.

As has been described above, some embodiments of the invention comprise a plurality of sensors on or near a shaped device on which a body tissue may be fixated or arrayed for treatment. In some embodiments, one or a plurality of such sensors may be used to optimize delivery of energy to target tissues. As may be seen in FIG. 6B and as discussed above, a controller such as controller 610 may use an iterative method to aim energy from an energy source outside a treated organ (and optionally outside the body) towards target tissues. As explained above, in some embodiments, in a measurement phase, controller 610 directs detectable but optionally non-destructive energy from emitter 604 towards sensor(s) having a known (or predicted) spatial relationship to those target tissues. Subsequently, in a treatment phase, more powerful energy may be delivered into the target tissues using aiming parameters calculated during the measurement phase. (It is noted that in some embodiments, measurement phase activities and treatment phase activities may be combined in other than the cyclic manner described above.)

FIG. 6B shows an optional process for perfecting this aiming of energy. Optionally, tissue modulating system 600, as discussed above in relation to FIGS. 5 and 6A may be used to aim energy delivery towards a target tissue using methods described in FIG. 6B and FIG. 7B.

As shown in the figure, once a sensor 504 has been positioned within a patient at 702 (for example, using devices shown in FIGS. 4A and/or 4B, or similar devices), an energy beam (optionally of low and non-damaging intensity) may be aimed at an estimated target position, or may be randomly aimed somewhere in the vicinity of the target, as shown at 704. Then, according to an optional exemplary method shown in FIG. 6B between 706 and 708, a controller 610 may control scanning of an energy beam from an emitter 604 along Cartesian dimension (i.e. x, y, and z dimensions) one after another, in each case moving the beam, optionally in a step-wise fashion, and comparing signals received at a sensor 504 for each iteration with signals received at a previous iteration. (Note: reference to Cartesian coordinates is exemplary and not limiting. Calculations may be made with reference to spherical dimensions and/or with reference to any other methods of measuring and controlling direction of a beam.)

From this information controller 610 identifies aiming parameters which produced maximums (or desired minimums, or any other desired characteristic) of energy delivery as measured at sensors 504 according to some criterion such as intensity or a desired phase relationship. This process is optionally repeated separately for each dimension, or alternatively aiming parameters for more than one dimension can be evaluated by controller 610 in a common process.

Once aiming parameters producing desired maxima are determined as described above during a measurement phase of activity, treatment shown at 710 may take place optionally using a more intense energy beam, in a treatment phase of activity. Optionally, this process may be repeated as shown at 712.

In some embodiments, calibration data known to controller 610 (i.e. from calibration experiments performed prior to patient treatment) may provide information enabling controller 610 to aim an energy beam a controlled distance away from a sensor in a selected direction. Additionally or alternatively, controller 610 can calculate a difference between aiming parameters required to aim a beam towards a first sensor 504 and aiming parameters required to aim a beam towards a second sensor 504, and interpolate and/or extrapolate to calculate aiming parameters for aiming a beam at a target position having a known or predicted spatial relationship with the first and second sensors. Additionally or alternatively, controller 610 may use more than two such sensors for increased accuracy and/or to provide three-dimensional measurements. In some embodiments such a plurality of sensors are mounted so as not to be all co-planar, thereby facilitating gleaning three-dimensional measurements therefrom.

In an optional similar alternative method, controller 610 may count a number of iterative aiming steps (or the quantitative changes in energy projection parameters) required (e.g. for each Cartesian dimension) to move an energy beam's point of focus from a first sensor position to a second sensor's position, and used this information, using known mathematical methods such as for example interpolation and extrapolation, to calculate energy projection parameters required to aim an energy beam at a selected displacement away from the sensors. For example, in some embodiments beam displacement per iteration may be measured by dividing a known distance between two sensors by the number of iterations required to move a detected energy maximum from the position of a first sensor to the position of a second sensor, and use that information to calculate desired aiming parameters for a treatment phase of operation.

In some embodiments, controller 610 may aim treatment energy towards a controlled displacement away from sensors 504, to protect the sensors. Similarly, in some embodiments, controller 610 may aim treatment energy towards a controlled displacement away from sensors 504, according to known or predicted information regarding the spatial relationship between a target tissue and a sensor 504. For example, one or more sensors 504 may be positioned on or adjacent a shaped device (e.g. as shown in FIGS. 4A and 4B) which is inside a blood vessel, and controller 610 may aim an energy beam towards tissues (e.g. afferent or efferent nerves of the kidney) which are positioned near sensors 504, but outside the blood vessel and/or on an portion of the blood vessel proximal or distal to the position of the sensors.

Information which relates changes in energy projection parameters to detectable changes in positioning of delivered energy beams in the vicinity of the target, determined as described above, may also be used to provide energy in a planned or predetermined pattern over a tissue surface, such as for example over the surface of a shaped device to which a target tissue is fixated. In other words, in optional embodiments a series of beam-aiming parameters is calculated and successively used to displace a tissue-modulating energy beam in a pre-planned pattern over an extended surface of a target tissue. In other words, processes shown in FIGS. 6B (and 7B, discussed below) enable an energy projection system to ‘walk the beam’ over a target tissue surface in controlled directions and for controlled amounts, optionally while modifying beam position to follow target tissue movement. Optionally, this pre-planned pattern may be planned to treat a first organ while also avoiding exposure of a second organ to damaging energies.

In some embodiments, a set of beam parameters is measured during treatment, and a processor (such as controller 610 or controller 1610) is programmed to detect beam characteristics (such as phase, amplitude, etc.) sensed by sensors which substantially differ from expected characteristics (such as, for example, beam characteristics measured in advance during calibration, or beam characteristics predicted by calculations). If beam characteristics substantially different from expected values are detected, the processor/controller may halt energy projection and/or notify an operator and/or initiate a preemptive measurement phase as described above, to better align beam parameters (such as position and amplitude) to actual tissue movements, or to otherwise correct beam aiming and control.

In some embodiments of the invention, a system controls the position of the beam by conducting a single measurement cycle at the beginning of treatment, and conducts continuous treatment, until measurement indicates a possible movement of tissue; this is especially beneficial for target tissues which are not expected to be displaced during treatment, and when duty cycle of the treatment phase may contribute to efficacy and/or reduce total duration of treatment.

It is an important advantage of some embodiments of the present invention that systems and methods here described enable delivery of aimed and controlled doses of projected energy, without requiring imaging of tissue nor imaging of the energy beam. This may be contrasted with prior art methods. For example, comparatively complex and expensive and low-resolution MRI scanning has been used to detect heat induced in body tissues by an energy delivery process, and this information has been used to enable corrected aiming of a treatment beam.

As shown in an exemplary embodiment shown in FIG. 6B, in some embodiments, aiming parameters for delivering maximal energy to a selected target are found by maximizing energy readings at the sensor(s) while iteratively moving the beam one step at a time along one dimension, then doing the same for each of the other two dimension. However, it should be understood that this method of aiming is exemplary and not to be considered limiting. Any other scanning method may be used to determine energy maxima and sharpness of focus. For example, a ‘first approximation’ scan using large iterative steps might precede a fine scan using smaller iterative steps, so as to speed the scanning process. Similarly, use of information about maxima found in a previous iteration and/or predictive movement analysis based on cumulative information (e.g. as a patient repeatedly breathes) may be used to facilitate rapid iterative scanning of a moving target such as a tissue which moves when a patient breathes.

Note that although the above discussion of FIG. 6B refers in general to “maximizing” energy delivery, maximization of energy amplitude is only one of many possible optimizations which may be accomplished using the method shown. “Better” and “worse” as those terms are used in FIG. 6B may optionally refer to any criterion or combination of criteria desired by an operator. For example, one might use the method to minimize energy delivery to a sensitive area and/or one might select for a desired combination of amplitude and phase.

In some embodiments, at least three sensors are used, and these are optionally not aligned. Their relative positions may optionally be calibrated in advance and their relative positions stored in a memory and used in the calculations described above, and for additional aspects of optimization of aiming and/or patterned energy delivery. Optionally, the “better” and “worse” functions may be defined as a mathematical function of the amplitudes of three or more sensors, together with the position calibration data mentioned above, to provide a vector amplitude in 3D space. In some embodiments, location of three sensors, as is determined from calibration information as detailed above, is determined in each measurement phase, and a relative position of the target is determined using the local sensor's coordinate system. In other embodiments, treatment can be determined using a location of a single sensor, and directions which are determined by the position of the transmitter. In yet other embodiments, two sensors enable detecting an axis of treatment, and path of treatment is conducted relative to this axis using spherical coordinates.

Attention is now drawn to FIGS. 7A and 7B. FIG. 7A is a simplified schematic of a system 1000 for directing a beam at and/or near a sensor's location, useful when beam is generated by multiple transmitters such as phased array transmitters, according to some exemplary embodiments of the present invention. FIG. 7B is a simplified flowchart showing a method using system 100, according to some embodiments of the present invention.

System 1000 uses a controller 1610 in a manner similar to that described above with reference to FIGS. 6A and 6B, with the difference that emitter 604 comprises a plurality of emitting elements 2604 (also referred to herein as transmitting elements 2604), and that controller 1610 discovers and uses the relationship between beam generation parameters and energy received at one or more sensors 504, for each transmitting element 2604 separately and/or for small contingent groups of transmitting elements 2604, such as the shaded group 1030 shown in the figure. Optionally, emitter 604 may be a phased array 1604, for example a HIFU phased array 3604.

For example, during a measurement phase of activity (which may alternate with treatment phases of activity in a cyclical manner as described above), controller 1610 fires each transmitting element 2604 (or small group of elements 1030) in turn, and measures time of flight for that element (i.e. measures the delay between firing and detection of the energy at a sensor 504. These measurements may then be used as described above to adjust transmitting parameters (including timing of firing) for some or all elements 2604, optionally so as to coordinate and/or synchronize the effects of firing of elements during the treatment phase of activity to create a desired effect such as, for example, the effect that beamed energy from all transmitting sources arrives at a same target simultaneously and in phase. FIG. 7A shows a target 1010 optionally fixated to a shaped device 502 which comprises a plurality of sensors 504.

Optionally, controller 1610 makes timing/phase/intensity measurements for individual transmission elements 2604, and/or for small sets or groups of elements 1030. For example, if power available for a single element is not sufficient for sensing, a set of 9 (3×3) elements, (or 16, or 25, etc., or some other grouping of elements) may be fired concurrently with a same phase, and used to detect the time of flight from the middle element to each sensor. (The physics of beams enables measuring a single reading from each sensor as representing the time of flight of the middle element, despite the fact that the set of elements fires at once.)

Calculating the time of flight to each sensor, and knowing the relative time of flight between every sensor to the others (either by measuring this in real time, or by laboratory calibration, or by theoretical calculation), using four sensors which are not co-planar, enables controller 1610 to determine the timing of a beam from each set of transmitting elements to a sensor and/or to a plurality of sensors, and therefore enables to determined the required time of flight to a required target point near the sensors.

It is important to note that in ultrasound waves, the time of flight depends on tissues in the sound path. In tissues such as human tissue, speed of sound can vary, in a same part of the body, by as much as 10% (for example, if a path between some transmission elements 2604 and a sensor 504 traverses the kidney, while a path from other elements to the sensor traverses fat.) The result of such a situation is that the energy beams can change direction, according to Snell's law. Therefore the direction from which a beam reaches the sensors is not necessarily a straight line between the sensor and the originating transmission elements, but rather some deflected direction. Therefore controller 1610, to calculate aiming parameters for each transmission element to be used in a treatment phase of activity, must take into account time of flight from each transmission element 2604, so as to calculating resulting actual beam directions and phase coordinations between beams originating in different transmission elements.

Optionally, the measurement phase may be accomplished using non-harmful energy levels. Optionally, measurements with respect to each element or small group of elements is of short duration. Controller 1610 may make measurements for all elements 2604 in each cycle, or alternatively may make measurements of only some elements per cycle. Measurements may include time of flight and/or phase and/or amplitude and/or any other measurement helpful for determining the transmission parameters of that element or elements. Optionally, controller 1610 and an array 1604 may use different frequencies or other signal modulations to distinguish between measurements of multiple elements tested simultaneously or near-simultaneously. Optionally, same frequencies are used to make common measurements for a plurality of nearby transmission elements.

Using the methods described above the system may determine which transmitting elements successfully transmit energy detectable by the sensors, and may determine what transmitting parameters (e.g. phase, amplitude) are required to make each element's energy transmission detectable.

After measuring all or many elements in transmitter 1604, controller 1610 may calculate global parameters (such as general distribution of power to transmitting elements, a phase of each element, etc.) required for transmission during a therapeutic phase of activity. Optionally, controller 1610 may prevent energy transmission during the treatment phase from elements 2604 discovered during the measurement phase to have been obstructed, their energies not reaching sensors 504. Blocking transmission from these elements during the therapeutic phase reduces risk of damage to obstructing tissues, and reduces energy transmitted during treatment.

Optionally, each measurement phase may measure only a subset of the transmission elements per each measurement cycle, for example if measuring all would require more time than is available during the cycle. According to this optional method, the system measures only a selected subset of elements each cycle, and may use such algorithms as interpolation and/or extrapolation to calculate the transmission parameters of the other elements not measured. Optionally transmitting parameters of non-measured elements may be calculated from neighboring elements and/or from prior cycles when the transmitting element was measured. When target tissue and sensors are far from the transmitting elements, it can take considerable time for each transmission to reach the sensors. For example, transmission from a transmitter 15 cm away from a sensor might take about 10 milliseconds to reach the sensor, whereas transmission from a closer transmitter would take less time to reach sensor and therefore, if the transmitters were fired at too close a time interval, a sensor might receive later signals from a closer transmitter before receiving or while receiving earlier signals from a more distant transmitter. Therefore, in some embodiments, distances from all transmitters to all sensors may be used to compute predicted delays, and during the measurement phase transmitters may be fired one after the other with at least a computed appropriate delay between transmissions, an appropriate delay being at least a delay which will avoid the possible confusion of order and/or overlapping received signals just mentioned.

In some embodiments, in order to be able to measure more transmitters at a single measurement cycle, the transmitters are fired in a physical sequence which makes sure each measured transmitter is very close geometrically to the prior one, thereby assuring that signals will arrive at sensor at the same order they were transmitted.

Attention is now drawn to FIG. 7B, which is a flowchart providing details of a method of aiming energy from a multi-element energy source 1604 (optionally a phased array 3604) towards a target tissue, according to some embodiments of the present invention.

In some embodiments, a method for aiming a phased array comprises, in a measurement phase of activity, iteratively firing a plurality of individual elements (or small groups of elements) of an array of elements, and measuring “time of flight” (the time required for energy from the fired array elements to be detected at one or more sensors. At 802, a phased array energy transmitter (such as, for example a HIFU ultrasound source embodied as a phased array) is positioned near a patient. In some embodiments a shaped device (such as those presented in FIGS. 4A and 4B, or any other shaped device) is used to fixate target tissues in a known form. In some embodiments, a sensor or plurality of sensors 504 mounted on or near the shaped device detect and report detection of energy provided from the energy transmitter.

In some embodiments, in an iterative process starting at 810, a first element or set of elements is fired at a known time, and one or more detectors 504 each report the time of detection of that energy as it reaches the vicinity of the treatment target. This process may be repeated for some or all of the array elements and for some or all of the available sensors. In this manner, the “time of flight”, the time required for the energy transfer between each element or small set of elements and each sensor in the target vicinity, may be measured and recorded. For example, elements of a phased array transmitter may be fired as bursts, for example 20 microseconds each, with some silent duration in between, for example of 20 microseconds each.

In some embodiments, sensors measure phase and amplitude. In some embodiments phase is used to calculate exact time of flight. In some embodiments a single frequency of transmission is used for measurement. In some embodiments multiple transmitted energies are fired and measured concurrently. In some embodiments multiple frequency of transmission is used sequentially. In some embodiments data from multiple frequencies is used to calculate time of flight.

FIG. 7B shows the iterative process terminating at 815 followed by a calculation at 820, but this is optional: alternatively, measuring, recording, and calculating may be performed together.

A processor or controller (e.g. controller 1610 of FIG. 7A), having access to that recorded information, may calculate (at 820) times of firing which will result in in-phase energy deliveries from a plurality of array elements to one of the sensors and/or to a position in the tissue having a known spatial relationship to the sensors. At 830, using interpolation, controller 1610 may extend that calculation to elements whose ‘times of flight’ were not explicitly measured. At 840, in a treatment phase of activity, controller 1610 fires elements 2604 in a calculated timing pattern to produce in-phase energy delivery at desired positions. As described above with respect to FIG. 6B, low (non-destructive) energies may be used during the phase in which ‘times of flight’ are being measured, and more powerful tissue-modulating energies may optionally be used when the timing calculations have been completed and the phased array starts firing energy according to the calculated timing pattern to treat the target tissues.

It should be noted that in the case of HIFU the energy being delivered is acoustical. Therefore a) the energy may be blocked by certain types of intervening objects (bones, gas, etc.), and b) the energy delivery will be subjected to unpredictable differences in transmission speeds, depending on the exact density and other physical characteristics of the body tissues and other material through the acoustical energy passes on its way from its originating phase array element towards any particular tissue in the target vicinity.

With respect to (a), the timing detection process (between 810 and 815 on the figure) will detected instances where energy projected from particular elements of the array fails to reach the vicinity of the target and is undetected (or detected as weakened) by the sensors in the target vicinity. In some embodiments, at 825, elements known to be momentarily incapable of sending energy to a particular portion of a target tissue (e.g. because of intervening acoustically opaque matter) are optionally shut down so as to protect intervening tissues or other objects whose presence in the energy path caused the energy delivery failure, to enable precise delivery of the other elements without the effect of scattering of these obstructed element beams, and to enable precise calculation of the total energy that reaches target. In some embodiments, transmission energy is altered as a result of discovery of such an obstruction, so that the total energy reaching target conforms to a desired level. In some embodiments, the duration for which a certain beam position is maintained is determined according to preplanned dose requirements as these relate to delivered energy as calculated as a function of detected energy received at sensors, as discussed above.

With respect to (b), it is an important advantage of some embodiments of the invention that timing differences in the transmission of the acoustical energy from individual elements of the array to portions of the vicinity of the target tissue are measured directly, and may consequently be taken into account in the calculations at 820. Sending energy pulses from the various elements of the array at times programmed to take into account the expected ‘time of flight’ of energy from any particular array element to a particular target region enables delivery of a clean and relatively focused energy signal, optionally timed to arrive in phase at a target, despite the fact that the speed of the acoustical energy through various body portions intervening between the energy source and the tissue target may differ significantly.

Optionally, measuring of time of flight may alternate with delivery of treatment energies in a cyclical iterative process, each cycle comprising a measurement phase of activity and a treatment phase of activity. Optionally, the timing measurements use a non-destructive level of energy as compared to the treatment phase energies.

Optionally, timing information gleaned as described above may be used to calculate timing of pulses from the phased array in a manner which results in a delivery of a pre-planned pattern of energy over a selected portion of a target vicinity, for example across the surface of a tissue fixated to a shaped device as described above.

During a measurement phase of activity, energy may be transmitted from a plurality of transmitting elements. According to some embodiments, during measurement phases of activity controller 1610 distinguishes between energies originating simultaneously at a plurality of sources by modulating energies transmitted from at least some of said sources, and detecting said modulation in energies detected by the sensors, thereby identifying a transmission source from which a modulated energy originated. In some embodiments, the modulation is a frequency modulation. In some embodiments, a plurality of transmission sources are fired in a known sequence, and information received at the sensor(s) is associated with sources according to the order in which the energies are detected. In some embodiments energy received at all or some sensors is measured during treatment cycles, and the system determines if the sensor readings are in expected ranges relative to target beam position. If detected energies exceed a pre-configured threshold, the system may conduct a non-planned measurement phase, so as to correct beam position.

In some embodiments, the system conducts a continuous treatment phase while monitoring sensor readings as described above, and conducts measurement cycles only when detected energies differ from expected values by more than a pre-defined threshold amount. These embodiments may optimize treatment time and energy dose.

It is noted that once beam transmission parameters are known for sensor/s, controller 1610 may use such methods as interpolation and/or extrapolation of parameters to direct a beam at target positions. Optionally, controller 1610 tracks beam parameters required to aim towards each sensor at multiple times during treatment, and uses interpolation and/or extrapolation, or other algorithms, to make a first guess at hitting a sensor, and then conducts a search around that location to find exactly what beam parameters are required for hitting that sensor at its current position. The system preferably conducts such an update procedure cycles short enough to enable smooth tracking of beam parameters even when the sensors are continuously moving, as, for example, when affected by the breathing or heartbeats of a patient.

It may be noted that the geometric position of treatment paths may be pre-configured relative to the sensor(s) and not relative to an external reference frame. This enables continuous aiming of transmission parameters without the need to measure any absolute positions of sensors, targets, or beams, relative to any imaging reference frame. Use of some embodiments here described does not require imaging during treatment. Some embodiments thus enable treating even tissues which cannot be imaged.

One aspect of the present invention is the capacity to treat a preplanned shape with known spatial relationship to a set of sensors, without using any 3D imaging modality even during preparatory phases of treatment. As discussed above, a sensor or set of sensors may be placed at or near a treatment target, optionally using a shaped device, so that a desired treatment shape is defined relative to the sensors. Placement of the sensors may be controlled by using a device which is optionally a luminal shape device within the body, such as catheter. Placement of the device may be assisted by any relevant type of imaging, such as x-ray in a cath-lab, but does not necessarily require 3D imaging. For example, positioning of sensors may be done using an intravascular catheter inserted into a patient in a cath-lab, using fluoroscopy, a 2D imaging modality.

Controlling each transmission element by itself (or in small groups of elements which are close to each other also enables shutting down elements that are obstructed (for example by bone, or air). This method is particularly useful in ablation of heart tissue using extra corporeal transmitters, in which some of the paths between element and target are temporarily or permanently blocked during treatment by ribs, esophagus, or other body features. Therefore the present method, in some embodiments thereof, eliminates need for a preoperative step of modeling the beam treatment using exact imaging, and enables the treatment of areas that have multiple obstructions and aberrations (in some cases even moving obstructions and changing aberrations), thereby providing a treatment which cannot be accomplished using prior art techniques.

An additional aspect of the present invention is a system and method for holding sensors (e.g. sensors 504) in place, for guiding a distal beam treatment.

A system may have one or more such devices, each placed in a different setting of the body, to enable elaborate geometrical definition of a treatment path. FIGS. 4A, 4B, and 5 provide examples of systems having a single intravascular catheter for renal denervation. Such a catheter may be inserted in a renal artery, may optionally be parked at a relatively straight portion of the artery, and a treatment path (i.e. energy path) may be defined with respect to the device. Optionally the device may be a shaped device operable to fixate the position of target tissues, as described above. In some embodiments, the shaped device may be operable to be located near or contiguous to a target tissue and immobilized with respect to that tissue.

Optionally, more than one device may be used. For example, for renal denervation, a first catheter comprising a sensor and optionally comprising a shaped device may be inserted in a renal artery, and a second catheter comprising a sensor and optionally comprising a shaped device may be inserted into a vein. This method enables treating tissue in the shape of an “8” formed around both vessels.

Another example is a trans-urethra catheter comprising one or more sensors. Used with the beam-directing methods described herein, such a catheter enables delivery of energy in a circular or tubular ablation pattern around a urethra, for treatment of BPH.

Tissue-fixating shaped devices and/or energy-detecting sensors may be used to defining the shape of a treatment path in relation to the shape of the devices, as described above, but such devices and sensors may also be used to guide an energy treatment so as to avoid hitting and damaging sensitive tissues. For example, some embodiments of the invention comprise a renal denervation system having two catheters, one catheter inserted in an artery and used to guide a treatment path around the artery, and a second catheter inserted into a vein and used detect energies which might damage the vein, and thereby used to assure that the treatment path around the artery does not penetrate the vein. An additional example is a trans esophageal catheter, inserted through the month or through a nasal cavity and having a distal end balloon or other expandable device in which one or more sensors are installed, used together with an intravascular catheter inserted into the heart for guiding electro physiological ablation of arrhythmia such as Atrial fibrillation. In some embodiments, an intravascular catheter comprising one or more sensors is inserted into the left atrium and positioned in the pulmonary vein, and a trans-esophagus catheter with a distal end balloon comprising one or more sensors are used together with an extra corporeal ultrasound transmitter. Sensors from both catheters and the ultrasound transmitter are connected to a computerized system, which controls beam parameters for conducting a pulmonary vein isolation procedure, using measurement information provided by sensors from both catheters. In some embodiments, one or both of the intravascular and intra esophagus catheters comprise mechanisms for fixating and/or controlling the shapes of the lumen they are in and their position within the lumen. (In other words, they comprise “shaped devices” as that term has been defined and used herein.) Indeed, use of such “shaped device” mechanisms on these catheters may provide for safer and more effective treatment, with or without reference to included sensors. For example, a trans-esophageal catheter may be provided with an inflatable balloon which is magnetized or comprises magnetic elements, or comprises electrical wiring enabling electromagnetic attraction. With this catheter, a magnet (optionally an electro-magnet) enables an operator to pull or push the internal catheter from the outside of the body, thereby controlling positioning of the catheter and immobilizing the catheter during treatment. For example, a magnet outside the body may be operated to push the esophagus away from the treatment point. Increasing distance of the esophagus from the treated point reduces the likelihood of damage to the esophagus as a result of the treatment.

In another embodiment, a system using energy measurement methods described above, analyzes the distance between the treatment target which is the intended focal point of an energy beam, and the esophagus, and uses that information to control the energy level of the beam as a function of that distance, thereby avoiding damaging the esophagus. Control of the energy beam intensity as a function of the distance of the esophagus may optionally be used together with methods for controlling the position of the esophagus, for example the magnetic method described above.

Control of internal distance between an organ and a treatment point may also be used to temporarily alter the shape of the organ to facilitate treatment. For example, a device such as those presented in FIGS. 4A and 4B may be used to adjust an artery shape to be round or to be in some other regular or pre-determined shape, to facilitate creating an accurate energy path around it so as to safely denervate its nerves without damaging the artery.

Such a mechanism for control of shape and/or position of an organ in order to avoid harming it, (with or without sensing and/or calculating distance to a treatment point) may also be beneficial when the treatment modality is not distant to target. For example, a catheter (e.g. an RF catheter), used to ablate pulmonary vein's entrance to the left atrium, is required not to damage the esophagus during treatment. A trans-esophageal catheter inserted in the esophagus and having a distal structure enabling it to be moved, preferably from the outside of the body (e.g. using the magnetic method described above) may be inserted through the month, or alternatively through the nose, (enabling treatment without anesthesia). Alternatively, or additionally, the catheter may have means to detect its distance from the intravascular catheter used for treatment of the heart, the distance detection being done by imaging, or by magnetic sensing of its position, or by measuring the beam intensity of an intravascular beam generator (such as RF, or ultrasound, etc.), or by sensing an extra corporeal beam energy. A system comprising such a sensing mechanism may alert a physician of increased risk if the treatment point in the heart is detected as coming too close to the esophagus. Once alerted, the physician (or optionally an automatic mechanism) may move the esophagus away from the treatment point, for example by manipulating external magnets which influence a magnetic element in an esophageal balloon catheter as described above, or by using internal pressure in an esophageal balloon catheter to modify the shape of the esophagus, or both. Such a system may modify the position of the esophagus and/or its shape while treating the heart or other organ, optionally moving the esophagus away from each point of ablation, as the ablation procedure proceeds from one ablation point to another. Optionally and additionally, such a catheter may have a temperature sensor or sensors to measure the esophagus temperature and warn a physician and/or automatically halt treatment when heat is detected to exceed a predetermined level. Optionally, such a system may also comprise a cooling mechanism such as irrigation, for cooling the esophagus while the heart is treated.

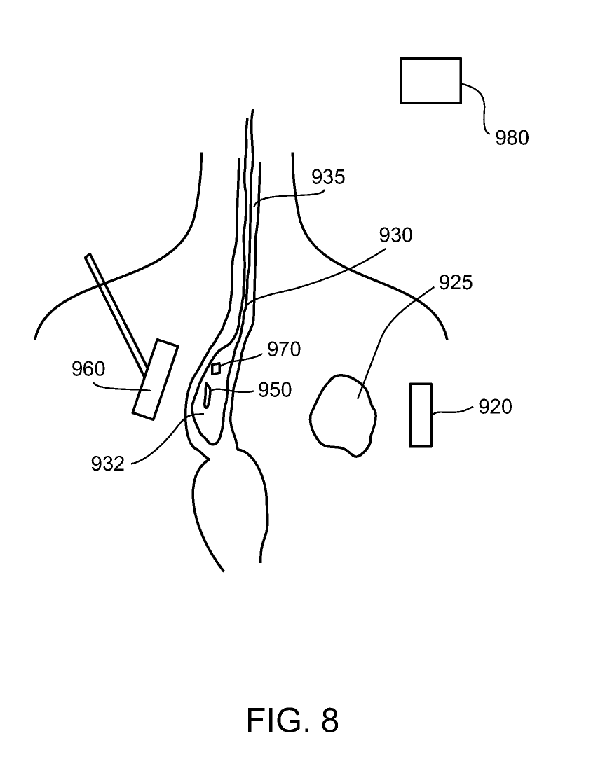

Attention is now drawn to FIG. 8, which is a simplified schematic showing an apparatus and method for protecting a first organ during treatment by a directed energy beam of a nearby second organ, according to an embodiment of the present invention. FIG. 8 shows an exemplary embodiment in which an energy projector 920 (optionally a HIFU projector) projecting energy towards a portion of a first organ, heart 925. (The figure is not drawn to scale.) A catheter 930 (optionally a balloon catheter having an inflatable balloon 932) is inserted in a second organ, an esophagus 935 in this exemplary embodiment. Optionally a balloon 932 is inflated within the esophagus. Catheter 930 comprises a magnetic or electromagnetic element 950 (optionally installed on or in balloon 932) capable of being attracted by a magnet or electromagnet 960. As described hereinabove, catheter 930, inserted in the esophagus, may be pulled magnetically (and/or pushed magnetically) so as to move it from its normal position near a heart, while that heart or a portion thereof is undergoing energy therapy (optionally by energy beam projection from projector 920), thereby protecting the esophagus from being damaged by that energy therapy. An optional sensor 970 provided on catheter 930 may be used to detect energy from projector 920 reaching sensor 970. Upon detection, a system controller 980, receiving signals from sensor 970, may warn an operating physician and/or stop or modify beam projection from projector 920 and/or move or activate magnet 960, thereby protecting esophagus 935. Similar systems and methods may be used to protect other second organs during energy treatment of other first organs.

In some embodiments, a system according to an embodiment of the present invention uses aimed energy beams in a planned pattern of energy delivery which treats a first organ while avoiding contact with a second organ, for example treating a heart arrhythmia while avoiding damaging an esophagus. The system may comprise a sensor mounted on a shaped device operable to fixate target tissues in a fixed position relative to the shaped device. The system may further comprise a mechanism for displacing said shaped device and a tissue fixated thereon, within a body, and/or for displacing a second organ to distance it from a first organ during treatment. The mechanism optionally comprises a magnet. Optionally, a portion of the mechanism is sized and shaped for insertion into an esophagus, optionally for insertion in a body through a mouth or through a nasal canal. The system may comprise an inflatable balloon or other expandable device and/or a sensor which detects heat and/or a sensor which detects beamed energy. In some embodiments the system comprises a controller which receives signals from the sensor and is programmed to halt and/or to modify an energy treatment upon receipt of a signal from the sensor reporting a condition which may be dangerous to the second organ.

In some embodiments, the second organ is a blood vessel, and the system comprises a mechanism for displacing the vessel, e.g. to avoid damage to the vessel during treatment of a first organ. The displacement mechanism optionally comprises a magnetic element insertable into the vessel, and a magnet exterior to the vessel, and optionally exterior to the body.

### Advantages of Some Embodiments as Compared to Methods of Prior Art:

Use of extracorporeal energy transmission for modulation of tissue has been known for many years. In order to treat target tissue, and cause minimal damage to non-target tissue, a system of such modulation capacity requires three basic capabilities: the ability to locate the target tissue position, the ability to define the 3D shape of the treatment required, whether a point, a path, or a volume, and the ability to know where the energy effective area such as its focus is located. When the target tissue moves, additional capacity, for tracking the tissue location is required.

Prior art describes multiple volumetric imaging modalities to locate where the target tissue is, and to determine target shape; such modalities include MRI, CT, PET and 3D ultrasound. These modalities are sometimes capable of imaging the target shape in 3D and its location, and imaging the treatment. These above mentioned systems however have some deficiencies: MRI is an expensive and time consuming technology, and requires additional technological and technical effort in not exposing magnetic instruments to the treatment theatre. CT uses ionizing radiation intensively. As such it is less favorable for imaging; for the same reason, CT is not suitable for dynamically tracking moving tissue targets, and therefore can be used only when organs are not expected to move, or when a patient is exposed to intensive radiation for treatment. PET has similar limitations as CT because it is usually used in a CT environment. Ultrasound imaging is limited in spatial resolution when imaging deep tissues; additionally, due to speckles and other artifacts it is difficult to identify shape accurately. Prior art suggests placing beacons, fiducial points or markers placed at or near target prior or during treatment, to enable better imaging of the target location using the imaging modality, or to correlate a preplanned treatment plan to the image. In such art, a beacon fiducial point or marker which is clearly visible in the imaging modality of choice, is placed on or near a target, and can be accurately imaged by the imaging modality, to enable treatment guidance. This method however is limited as it does not allow for detecting where the beam is, but only where target tissue points are. Other prior art examples include other means of detecting a position of a device placed at or near position of the target tissue, such as a magnetic position sensor placed on target, and a calibrated location system which enables to place the identified location on the image. These methods however do not achieve better knowledge of where the beam's effective area is located, and therefore lack the capacity to correct beam aberrations caused by moving through inhomogeneous tissues.

In order to know where the beam distal end or focus is located during treatment, prior art describes two distinct approaches: the first, uses prior knowledge (calibration data) of the beam position in correlation to its steering mechanism, as is measured in bench tests during calibration or pre calculated. Calibrating the imaging reference frame to the treatment beam reference frame enables to estimate where the beam would be in the imaging modality of choice, and enables to synthetically add this beam position to the image. This method is more fit to beam modalities that have minimal change of their geometric behavior in real-time relative to the bench test (e.g. X-ray radiation technology), and is also more fit for procedures where target tissue and vicinity are imaged properly during the procedure. This method however generates position errors when the beam path is distorted by tissues (e.g. when using ultrasound technology); in these situations in order to not risk healthy tissue damage due to position inaccuracies of the beam, a system requires a means to detect where the beam is during treatment, in real-time.

A second approach to knowing the location of the beam is measuring beam position in real-time. Prior art describes imaging means of locating the real time position of the beam during treatment; using such techniques for tracking beam position in real time depends on the beam modality of choice: X-Ray beams can be imaged using CT, but cannot be imaged by ultrasound. Similarly, ultrasound beams can be imaged using advanced MRI techniques, or advanced ultrasound techniques, but both lack spatial and/or temporal resolution. Other means of detecting a beam's location use the beam's effect on the tissue as an indication, for example by measuring heated areas (in MRI guided treatments) or detecting lesions made by the beam (in both CT, MRI, and ultrasound guided treatments). Obviously, such techniques have the drawback that they are unable to detect the beam position without causing harm to the tissue the beam hits.

An additional disadvantage of prior art technologies occurs when a target location is far from an energy transmitter used to treat it. Successful treatment (e.g. by HIFU) normally requires a large aperture transmitter. Yet in some clinical cases the (usually conical) path from transmitter to target is partially obstructed by other tissues. Ultrasound beams, for example, may be obstructed by bone or air (e.g. in lungs or in the digestive system). In these situations it would be beneficial to modify the transmitted energy in a way that would limit energy transmission from transmitter portions which are obstructed, both because they are not effective and because they may damage the obstructing tissues. For example, in a transmitter made with phased array technology (e.g. phased array ultrasound) it would be beneficial to shut down array elements of the transmitter which are obstructed from reaching target. This challenge is increased when the treatment area is deep, enforcing a large transducer; such clinical situations include, for example the treatment of renal nerves surrounding the renal blood vessels. Treatment at a distance requires a large transmitter, portions of which may be obstructed by ribs or lungs or intestines. When the target tissue moves, for example because of respiration, the obstructed areas of the transmitter dynamically change their shape and position during treatment. Imaging of beam position, as practiced by methods of prior art, is unlikely to enable detection of such obstruction in real time, as the energy of obstructed beam is scattered and limited in power.

It is another limitation of prior art systems that 2D imaging cannot correlate the position of a beacon, or fiduciary point, or marker, for moving targets: since the image of a plane (for example an XY plane) cannot be taken simultaneously with another plane (for example XZ plane), using image guidance for the treatment of moving tissues is not realistic with 2d imaging modalities such as simple (non CT) x-ray, fluoroscopy, or 2d ultrasound, using prior art techniques. Yet in many clinical cases, respiration and other movements cause treatment targets to move during treatment.

It may be seen from the above that systems and methods presented herein provide a variety of advantages over the systems and methods of prior art.
Systems and methods described, in some embodiments thereof, enable to treat tissue using only those elements of a phased array whose energies are actually detected at a vicinity of the target tissues.
Methods described herein, in some embodiments thereof, are advantageous in that they do not require an active imaging modality during treatment, and therefore reduce requirements for types of modalities used. For example, some methods here described enable use of a fluoroscopic to guide placement of sensors, eliminating need for CT or MRI, which are relatively expensive and difficult to work with.
Some methods described herein also enable a more precise positioning of the beam relative to target than is available using methods of prior art, as they do not require detecting beam positions with respect to an imaging reference frame, and do not require registering of points (such as beacons or fiducial points) in a prerecorded session, as required by some methods of prior art.
An additional advantage of some methods described herein is that they provide means to overcome distortions due to inhomogeneous tissues on the path of the energy beam: since each element or small subset of elements is measured separately, measurements of time of flight and received intensities are sensitive to differences in the speed of sound in tissues through which the energy beams must pass, so that calculations of the timing and phase and intensities to be used at the various elements during the treatment phase automatically take transmission differences imposed by tissues intervening between transmitters and target into account. This method is specifically beneficial for aiming beams where obstruction and aberration are high, like brain treatment (aberration by skull) or any treatment of tissue through the ribs, or air (such as like lungs or intestines). This method is particularly useful in any situations in which large timing differences occur, and where using prior art methods to aim a whole beam at a single “geometrical” position would smear the location of the focal area of the beam, making it both unsafe and ineffective.
An additional advantage of methods described herein, in some embodiments thereof, is that they enable aiming of therapeutic treatments at moving targets without requiring imaging.
Methods described herein, in some embodiments thereof, enable to direct a beam to target tissue without knowing either beam position or tissue position relative to any reference frame. As discussed, sensors placed at or near target tissue may be used to aim treatment beams toward treatment targets. Optionally, these methods may be used to treat an extending target according to a pre-planned treatment pattern, using aiming methods discussed above, aimed with reference to sensors whose position relative to a position of a pre-planned treatment target pattern are known. This method replaces the need for knowing target position relative to any external reference frame; this method uses a single type of sensor to analyze beam parameters and determine required beam parameters for reaching required position, without the need to know beam position relative to an external reference frame, and without the need to image the beam or its effects. In some clinical situations the shape of a treatment target is known in advance; these include shapes which have been preplanned using 3D imaging prior to treatment (for example liver cancer treatment, where the tumor shape is analyzed using CT or MRI and a shape of treatment is preplanned by an operator or automatically, or a combination of both), or when the anatomy of the treatment shape is known in advance (such as the treatment path surrounding renal arteries in renal denervation, or a pulmonary vein EP isolation in the heart surrounding the vein's entry point to the heart). The shape of the treatment path, being known, may be computerized without the need to image the treatment area, with reference to sensors whose positions with relation to the treatment target is known. It is noted that in some cases, such as renal denervation for example, prior art methods including imaging the treatment area would not reveal any information relevant to the treatment path, because some target tissues cannot be imaged. In the case of renal denervation, for example, the nerves cannot be imaged with any current imaging modality.
   Sometimes a treatment shape can be determined as being fixed relatives to the sensors (for example a fixed point in the brain relative to a vein in the brain). In other examples it may be determined as being parametrized, for example a path around an artery could use the artery's diameter for treating nerves surrounding the artery, for renal denervation. In other examples, a treatment shape may be preplanned using a 3d imaging modality prior to treatment. Aiming methods described above can send treatment energies to the planned shape, without need for imaging.
An Additional benefit of the present invention, in some embodiments thereof, is that they enable placing the required senses and devices within the body using relatively simple and inexpensive imaging modalities. Such positioning generally does not require any 3D imaging capacity, and placement of a sensor device is usually possible using 2D imaging modalities, such as for example a 2D x-ray or fluoroscope.

As used herein the term “about” refers to ±10%.

The word “exemplary” is used herein to mean “serving as an example, instance or illustration.” Any embodiment described as “exemplary” is not necessarily to be construed as preferred or advantageous over other embodiments and/or to exclude the incorporation of features from other embodiments.

The word “optionally” is used herein to mean “is provided in some embodiments and not provided in other embodiments.” Any particular embodiment of the invention may include a plurality of “optional” features unless such features conflict.

The terms “comprises”, “comprising”, “includes”, “including”, “having” and their conjugates mean “including but not limited to”.

The term “consisting of” means “including and limited to”.

The term “consisting essentially of” means that the composition, method or structure may include additional ingredients, steps and/or parts, but only if the additional ingredients, steps and/or parts do not materially alter the basic and novel characteristics of the claimed composition, method or structure.

As used herein, the singular form “a”, “an” and “the” include plural references unless the context clearly dictates otherwise. For example, the term “a compound” or “at least one compound” may include a plurality of compounds, including mixtures thereof.

Throughout this application, various embodiments of this invention may be presented in a range format. It should be understood that the description in range format is merely for convenience and brevity and should not be construed as an inflexible limitation on the scope of the invention. Accordingly, the description of a range should be considered to have specifically disclosed all the possible subranges as well as individual numerical values within that range. For example, description of a range such as from 1 to 6 should be considered to have specifically disclosed subranges such as from 1 to 3, from 1 to 4, from 1 to 5, from 2 to 4, from 2 to 6, from 3 to 6 etc., as well as individual numbers within that range, for example, 1, 2, 3, 4, 5, and 6. This applies regardless of the breadth of the range.

Whenever a numerical range is indicated herein, it is meant to include any cited numeral (fractional or integral) within the indicated range. The phrases “ranging/ranges between” a first indicate number and a second indicate number and “ranging/ranges from” a first indicate number “to” a second indicate number are used herein interchangeably and are meant to include the first and second indicated numbers and all the fractional and integral numerals therebetween.

It is appreciated that certain features of the invention, which are, for clarity, described in the context of separate embodiments, may also be provided in combination in a single embodiment. Conversely, various features of the invention, which are, for brevity, described in the context of a single embodiment, may also be provided separately or in any suitable subcombination or as suitable in any other described embodiment of the invention. Certain features described in the context of various embodiments are not to be considered essential features of those embodiments, unless the embodiment is inoperative without those elements.

Although the invention has been described in conjunction with specific embodiments thereof, it is evident that many alternatives, modifications and variations will be apparent to those skilled in the art. Accordingly, it is intended to embrace all such alternatives, modifications and variations that fall within the spirit and broad scope of the appended claims.

All publications, patents and patent applications mentioned in this specification are herein incorporated in their entirety by reference into the specification, to the same extent as if each individual publication, patent or patent application was specifically and individually indicated to be incorporated herein by reference. In addition, citation or identification of any reference in this application shall not be construed as an admission that such reference is available as prior art to the present invention. To the extent that section headings are used, they should not be construed as necessarily limiting.

1. A method of directing a tissue-modulating energy beam from a high intensity focused ultrasound (HIFU) projector distant from a target tissue to treat said target tissue in a body of a patient, comprising:
a) positioning within a body and near a target tissue at least one sensor operable to detect energy from a Phased array HIFU projector having a plurality of transmitting elements;
b) aiming towards a plurality of positions within an intrabody volume containing said at least one sensor a plurality of experimental energy beams of non-damaging intensity from said plurality of transmitting elements, each beam being aimed according to at least one experimental beam-aiming parameter used to aim said each beam;
c) selecting, from among said plurality of experimental beam-aiming parameters, a parameter shown by detection of beam energy at said sensor to have aimed its associated experimental energy beam towards said sensor; and
d) aiming a tissue-modulating treatment energy beam from said plurality of transmitting elements toward said target tissue according to a treatment-beam-aiming parameter calculated as a function of said selected experimental beam-aiming parameter;
wherein said Phased array HIFU projector distant from said target tissue is positioned outside a body.

2. (canceled)

3. The method of claim 1, wherein said aiming of said plurality of experimental energy beams comprises scanning said non-damaging energy beam over said intrabody volume by successively modifying at least one beam-aiming parameter to affect aiming of said experimental beams, while monitoring a phase and an amplitude of beam received at said at least one sensor.

4. The method of claim 1, wherein selecting said selected parameter comprises selecting from among said plurality of experimental beam-aiming parameters a parameter whose aimed beam produced within 5% of the maximum energy detected at said sensor for a group of experimental beams.

5. (canceled)

6. The method of claim 1, wherein said tissue-modulating beam is aimed towards a position at a calculated displacement from a position of said sensor.

7-8. (canceled)

9. The method of claim 1, wherein said sensor is an x-ray sensor and said Phased array HIFU projector is a source of x-rays.

10. The method of claim 1, further comprising identifying said beam-aiming parameter by successively modifying beam aiming along a first and then along a second Cartesian coordinate, and detecting separately for each Cartesian coordinate which beam-aiming parameters maximize energy received at said sensor.

11. The method of claim 10, further comprising modifying beam aiming along a third Cartesian coordinate, and detecting which beam-aiming parameters maximize energy received at said sensor.

12. The method of claim 1, further comprising identifying said beam-aiming parameter by successively modifying beam aiming according to first and then second coordinates in a spherical coordinate system and detecting separately for each coordinate which beam-aiming parameters maximize energy received at said sensor.

13. The methods of claim 1, further comprising detecting a delay between emission of said beam at said Phased array HIFU projector and detection of said beam at said sensor.

14. The methods of claim 1, further comprising cyclically repeating a first process which comprises aiming said experimental beams and selecting at least one of said selected parameters, followed a second process which comprises by aiming at least one tissue-modulating energy beam, in a repeating cycle.

15. The method of claim 14, wherein said projecting of experimental energy beams and selecting said selected parameters and occupies between 0.1% and 30% of each of said cycles.

16. The method of claim 1, further comprising utilizing differences between identified parameters of two different sensors at a known distance from each other to calculate a beam-aiming parameter which, when used to project a beam, will displace said beam from said sensors by a pre-calculated amount.

17. The method of claim 16, wherein said sensors are mounted on a catheter in a blood vessel, and said calculated treatment-beam-aiming parameters are calculated to direct a beam to a position near said blood vessel and distanced from said sensors.

18. The method of claim 14, further comprising cyclically repeating said first process and said second process to direct said tissue-modulating beam at a moving treatment target.

19-20. (canceled)

21. The method of claim 1, further comprising calculating a series of beam-aiming parameters which displaces said tissue-modulating beam in a pre-planned pattern over an extended surface of said tissue.

22-24. (canceled)

25. The method of claim 1, further comprising separately measuring a relationship between beam generation parameters and energy detected by at least one sensor, for each of said plurality of transmitting elements;
wherein a plurality of transmitting elements are transmitted at different times during a same cycle.

26. The method of claim 25, further comprising distinguishing between energies originating simultaneously at a plurality of sources by modulating energies transmitted from at least some of said plurality of transmitting elements, detecting said modulation in responses of said sensor, and identifying a transmission source from which a modulated energy originated according to said detected modulation.

27. The method of claim 26, wherein said modulation is a frequency modulation.

28. (canceled)

29. The method of claim 25, further comprising transmission a plurality of transmitting elements in a known order during a same cycle, and relating energy signals received at said sensor to particular originating transmission elements according to an order in which said energy signals are detected; further comprising measuring a delay between transmission of an energy beam and detection of said beam at said sensor, for a plurality of Phased array HIFU projectors.

30. (canceled)

31. The method of claim 29, further comprising transmission a plurality of elements of a phased array, said transmission occurring in a timed sequence whose timing is calculated based said measured delays.

32. The method of claim 25, further comprising using energy beams at said non-damaging intensity from each of said plurality of transmitting elements to identify those sources from which projected energy is detected by a sensor near said target tissue, and aiming said tissue-modulating energy beam towards said target tissue only from those Phased array HIFU projectors from which energy of said non-damaging intensity was successfully detected by said sensor.

33. (canceled)

34. The method of claim 1, wherein said plurality of beam-aiming parameters are calculated from a sequence of simultaneous transmissions each of one of said plurality of transmitting elements.

35. The method of claim 1, further comprising using a plurality of sensors at least some of which are located within different body organs.

36-40. (canceled)

41. A system for directing towards a target tissue in a body of a patient a tissue-modulating energy beam from an Phased array HIFU projector distant from said target tissue, comprising:
a) a Phased array HIFU projector having a plurality of transmitting elements distant from said target tissue;
b) at least one sensor operable to detect energy from said Phased array HIFU projector and positionable within a body in a vicinity of said target tissue;
c) a projection controller programmed to aim towards a plurality of positions within an intrabody volume containing said at least one sensor a plurality of experimental energy beams of non-damaging intensity from said plurality of transmitting elements of said Phased array HIFU projector, each beam being aimed according to at least one experimental beam-aiming parameter used to aim said each beam, to selecting, from among said plurality of experimental beam-aiming parameters, a parameter shown by detection of beam energy at said sensor to have aimed its associated experimental energy beam towards said sensor, and control said plurality of transmitting elements to project a tissue-modulating treatment energy beam from said plurality of transmitting elements toward said target tissue according to a treatment-beam-aiming parameter calculated as a function of said selected experimental beam-aiming parameter.

42. The system of claim 41, wherein said energy projector is outside the body of a patient.

43. The system of claim 41, wherein said controller is programmed to command projecting of non-destructive energies in a variety of directions while collecting information from said sensor during a measurement phase of activity, and to subsequently command projection of tissue-modulating energies during a treatment phase of activity, using tissue-modulating energy beam aiming parameters calculated as a function of said collected information.

44. The system of claim 41, wherein said tissue-modulating energy beam aiming parameters are calculated so as to aim said tissue-modulating energy beam towards said sensors during a treatment phase.

45. The system of claim 41, wherein said tissue-modulating energy beam aiming parameters are calculated so as to aim said tissue-modulating energy beam towards a position at a known distance and direction away from said sensor during a treatment phase.

46. The system of claim 45, wherein said aiming parameters are systematically modified during a plurality of treatment phases to aim energy in a pre-planned pattern having a known spatial relationship to said sensor.

47-60. (canceled)

61. The system of claim 41, further comprising a sensor operable to detect at least one of
a) dangerous levels of heat in an organ; and
b) dangerous levels of beamed energy beamed to a position in or near said organ; and
further comprising a controller which receives signals from said sensor and is programmed to modify an energy treatment upon receipt of a signal from said sensor reporting a dangerous condition.

62-65. (canceled)

66. The system of claim 41, wherein said controller is programmed to calculate a delay between projection of energy by said Phased array HIFU projector and detection of said energy by said sensor.

67-72. (canceled)

73. The system of claim 41, wherein said projection controller is programmed to operate said Phased array HIFU projector to transmit from said plurality of transmission elements in a timed sequence wherein each transmission from one of said plurality of transmission elements is delayed from a transmission from another of said plurality of transmission elements by in a period of between 10 milliseconds and 200 milliseconds duration.

74-78. (canceled)
